SEQUENCE LISTING (4 sequences)

SEQ ID NO: 1 - protein, 183 aa, Homo sapiens
GPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 2 - protein, 201 aa, Homo sapiens
MHHHHHHHHGGGGGIEGRGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 3 - DNA, 549 bp, Homo sapiens
ggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaggtttctctatcacaaatccaaatggaacataaccatggagtcctatgtggtccacaccacctatgatgagtatgccatttttctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgaga

SEQ ID NO: 4 - DNA, 603 bp, Homo sapiens
atgcatcaccatcaccatcaccatcacggtggaggagggggtatcgagggccgcggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaggtttctctatcacaaatccaaatggaacataaccatggagtcctatgtggtccacaccacctatgatgagtatgccatttttctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgaga

(19) United States
(12) Patent Application Publication
(10) Pub. No.: US 2018/0074070 A1
(43) Pub. Date: Mar. 15, 2018

(54) BIOMARKERS FOR PREECLAMPSIA

(71) Applicant: A1M Pharma AB, Lund (SE)

(72) Inventors: Stefan HANSSON, Lomma (SE); Bo AKERSTRÖM, Lund (SE); Magnus Göran GRAM, Oxie (SE)

(73) Assignee: A1M Pharma AB, Lund (SE)

(21) Appl. No.: 15/558,167

(22) PCT Filed: Mar. 16, 2016
(86) PCT No.: PCT/EP2016/055619

§ 371 Date: Sep. 13, 2017

(30) Foreign Application Priority Data
Mar. 16, 2015 (DK) PA 2015 70146
Dec. 3, 2015 (DK) PA 2015 70794

(51) Int. Cl. G01N 33/68

(52) U.S. Cl. CPC G01N 33/689; G01N 2800/368; G01N 2800/50; G01N 2333/805; C12Q 2600/158; G01N 2333/4713

(57) ABSTRACT

The present invention relates to the use of hemopexin, free, non-cell bound fetal hemoglobin and alpha-1-micro-globulin as markers for preeclampsia.

### FIELD OF THE INVENTION

The present invention relates to biomarkers for preeclampsia, for early onset (before 34 gestational weeks) preeclampsia and for late onset preeclampsia. Moreover, bo-markers for prediction of fetal and maternal outcomes in women suffering from preeclampsia are identified. The biomarkers are i) hemopexin (Hpx), Hpx in combination with alpha-1-microglobulin (A1M), or iii) HpX in combination with A1M and free circulating fetal hemoglobin (free HbF). The marker panel may be supplemented with other markers selected from haptoglobin-fetal hemoglobin complex (Hp-HbF), haptoglobin (Hp), heme oxygenase-1 (HO-1), and heme. CD163 and CD163 in combination with Hpx may be markers for fetal outcome. Both Hpx levels and activity (the latter denoted Hpx-a) may be used.

### BACKGROUND OF THE INVENTION

Preeclampsia (PE) complicates 3-8% of all pregnancies¹ and manifests clinically in the second half of gestation. The clinical characteristics that define PE are hypertension and proteinuria appearing after 20 weeks of gestation. PE is a potentially serious condition that if left untreated can lead to eclampsia, characterized by general seizures. A related disease, the HELLP syndrome, (hemolysis, elevated liver enzymes and low platelets count) develops more rapidly and is accompanied with maternal hemolysis. Uniform classification of the different forms of hypertensive conditions during pregnancy is important in order to be able to give a uniform diagnosis. To date several biomarkers have been suggested for screening in the first and second trimester, however none are yet used in clinical practice. Furthermore, some biomarkers have been suggested to support clinicians in their diagnostics and handling of the patients.

The pathogenesis of PE is not fully understood but recent studies have shown that extracellular fetal hemoglobin (HbF) is involved. Using gene expression microarray techniques and proteomics Centlow et al¹⁰ showed an up-regulation of the HbF gene and accumulation of cell-free HbF in the vascular lumen in term PE placentas. Later, Olsson et al¹¹ demonstrated that women diagnosed with PE have increased plasma levels of cell-free HbF and adult hemoglobin (HbA) and Anderson et al¹² demonstrated that the serum levels of HbF and A1M were elevated as early as 10 weeks of gestation in pregnancies destined to develop PE. It was hypothesized that HbF drives the generation of reactive oxygen species (ROS) and thereby induces oxidative damage to the placenta and a subsequent leakage over the feto-maternal barrier (including HbF). This overproduction and leakage of HbF result in an increased concentration of HbF in maternal plasma and further induction of ROS and inflammation. As a consequence, general endothelial damage leads to hypertension and proteinuria, the hallmarks of PE.

### DESCRIPTION OF THE INVENTION

One of the major constituents of blood is the protein hemoglobin (Hb), an oxygen-transporting protein that is packed in erythrocytes at high density. Hb is a tetramer consisting of four globin subunits each carrying a heme-group in its active center. In adults the most common Hb isoform is HbA, a tetramer that consists of two α- and two β-subunits (α₂β₂). In the fetus the HbF is predominant and consists of two α-chains and two γ-chains (α₂γ₂). Furthermore, heme consists of the organic ring-structure protoporphyrin IX that contains a ferrous (Fe²⁺) iron atom with high affinity for free oxygen (O₂). Ferrous Hb bound to O₂ is denoted oxyHb. Auto-oxidation of oxyHb is a spontaneous oxidation-reduction reaction eventually leading to production of ferric (Fe³⁺) Hb (metHb), ferryl (Fe⁴⁺) Hb, free heme and various ROS including free radicals. These compounds are chemically very reactive and have the potential to induce tissue damage and cell destruction by one-electron reactions with biomolecules.

Hb is normally found enclosed by the erythrocyte membranes. The auto-oxidation of intracellular oxyHb and downstream free radical formation is prevented mainly by superoxide dismutase (SOD), catalase and glutathione peroxidase (GPx). However, significant amounts of Hb escape from the erythrocytes under healthy conditions and massive amounts can be released during pathological conditions involving hemolysis. Therefore a number of defense mechanisms have evolved both in plasma and extravascularly to counteract the chemical threat of cell-free Hb to exposed tissues.

Haptoglobin (Hp) is perhaps the most well investigated Hb-clearance system. It binds cell-free Hb in plasma^{19,20} and binding to the macrophage receptor CD163²¹ clears the resulting Hp-Hb complex from blood. The Hp molecule consists of two chains, α and β, and two allelic variants of the α-chains exist, α1 and α2. As a result three phenotypic variants occur in the human population, Hp 1-1, 1-2, and 2-2. Free heme in blood is sequestered by hemopexin (Hpx) and the Hpx-heme complex is cleared from the circulation by the hepatocyte receptor CD91²⁵. In the intracellular compartment heme oxygenase (HO) is the most essential heme catabolic protein, converting heme to free iron, biliverdin and carbon monoxide (CO). The plasma- and extravascular protein alpha-1-microglobulin (A1M) binds and degrades heme and reduces metHb. A1M also acts as an antioxidant by reducing and covalently binding the downstream ROS and radicals generated by cell-free Hb and other sources.

Increased synthesis and accumulation of cell free HbF has been shown in PE placentas. Furthermore, increased concentrations of HbF have been shown in maternal plasma/serum in both early- and late pregnancy complicated by PE suggesting it to be an important factor linking stage one and two in the etiology. Free HbF has been shown to cause placental tissue damage and oxidative stress, which consequently leads to leakage over the blood-placenta barrier into the maternal circulation. To prevent toxicity of Hb and its degradation metabolites heme and free iron, several protecting scavenger systems protects the human body. Hp is the most well described Hb scavenging system that binds free Hb and transports it to macrophages and hepatocytes where its uptake is facilitated by the CD 163 receptor-mediated endocytosis. In the intracellular compartment of primarily macrophages Hb is degraded to heme by lysosomes, and heme is furthermore catabolized by HO-1 to biliverdin, CO and free ion. Biliverdin is then reduced to bilirubin, which is excreted via the bile system. CO has dilating effects on the vascular bed as it relaxes the smooth muscle layer of the vessels and consequently lowers blood pressure.

Hpx is a circulating plasma glycoprotein, mainly synthesized in the liver. It acts as an acute phase reactant and binds free heme with high affinity. The heme affinity to Hpx is affected by several factors, such as decreased pH, reduced state of the heme iron atom, binding of nitric oxide (NO) to the heme iron atom or presence of chloride anions and divalent metal ions. Sodium cations increase heme affinity to Hpx. The Hpx-heme complex is transported to macrophages and hepatocytes expressing the LDL receptor-related protein 1 (LRP1, also known as CD91), which facilitates uptake of the Hpx-heme complex. Hpx has indeed been shown to prevent endothelial damage in a mouse model. Besides heme-binding, Hpx also has other activities in plasma (Hpx activity). This includes enzymatic serine protease activity, inhibition of cellular adhesion, attenuation of inflammation and down-regulation of the angiotensin II receptor in monocytes, endothelial cells, and rat aortic rings.

Based on the results provided in the experiments reported herein, the present invention provides:
i) hemopexin and alpha-1-microglobulin as markers for preeclampsia, both for early and late onset preeclampsia,
ii) hemopexin, alpha-1-microglobulin and free, non-cell bound fetal hemoglobin as markers for preeclampsia, both for early and late onset preeclampsia
iii) hemopexin or A1M as a marker for preeclampsia, both for early onset and late onset preeclampsia,
iv) haptoglobin as a marker for preeclampsia and for late onset preeclampsia,
v) haptoglobin-fetal hemoglobin complex as a marker for preeclampsia,
vi) hemopexin and HO-1 (heme oxygenase) as markers for preeclampsia,
vii) a combination of hemopexin, haptoglobin, free fetal hemoglobin, and heme oxygenase as markers for preeclampsia,
viii) a combination of hemopexin, haptoglobin, free fetal hemoglobin, heme oxygenase and alpha-1-microglobulin as markers for preeclampsia,
ix) use of a combination of hemopexin and haptoglobin as markers for preeclampsia
x) use of a combination of hemopexin, haptoglobin and haptoglobin-fetal hemoglobin as markers for preeclampsia,
xi) use of a combination of hemopexin and heme oxygenase as markers for preeclampsia,
xii) use of a combination of hemopexin, haptoglobin, free fetal hemoglobin, and heme oxygenase as markers for preeclampsia,
xiii) use of a combination of hemopexin, haptoglobin, free fetal hemoglobin, heme oxygenase and alpha-1-microglobulin as markers for preeclampsia,
xiv) use of a combination of hemopexin-activity, hemopexin levels, haptoglobin, free fetal hemoglobin, heme oxygenase and alpha-1-microglobulin as markers for preeclampsia,
xv) use of any one of i)-v) together with free circulating fetal hemoglobin and/or together with alpha-1-microglobulin as markers for preeclampsia,
xvi) a method for diagnosing preeclampsia, early stage preeclampsia or late onset preeclampsia,
xvii) a method for evaluating progression or regression of preeclampsia, and
xviii) a method for assessing the effectiveness of a treatment of preeclampsia.
xix) use of any of the above together with a) fetal hemoglobin and/or b) alpha-1-microglobulin for assessing the effectiveness of a treatment of preeclampsia;
   in any of the above-mentioned settings, heme may also be included.

The above-mentioned markers and combination of markers may be used as predictive, prognostic and/or diagnostic markers.

The present invention also provides predictive biomarkers of a range of maternal and fetal outcomes:

i) free fetal hemoglobin and/or haptoglobin and/or hemopexin as predictive markers for predicting admission to neonatal intensive care unit (NICU)

ii) hemopexin as predictive marker for prematurity.

iii) use of any of i)-ii) together with alpha-1-microglobulin as predictive markers for predicting admission to NICU or prematurity

### Definitions

In this specification, unless otherwise specified, “a” or “an” means “one or more”.

Hemoglobin A (HbA). There exist several forms of Hb. Adult Hb (HbA) consists of two alpha and two beta polypeptide chains (Hbα, Hbβ), each containing a non-peptide heme group that reversibly binds a single oxygen molecule. Hb A2, another adult Hb component is composed of two alpha chains and two delta chains (Hbα, Hbδ).

Fetal hemoglobin (HbF). HbF, fetal hemoglobin, consists of two alpha chains and two gamma chains. The term “fetal Hb” refers to free HbF or any subunit of HbF and includes the HbF entities in a polypeptide (protein) or nucleotide (RNA) form, except when applied as a target for treatment. “HbF”, “fHbF” or “free HbF” refers to free fetal hemoglobin as defined below.

The term “free Hb”, in this specification refers to free Hb generally and includes total free Hb, free HbA, free HbA2, free HbF, any free Hb subunit (e.g. an Hbα, Hbβ, Hbδ or Hbγ chain), or any combination thereof. It further includes these Hb entities in either a polypeptide (protein) or nucleotide (RNA) form, except when applied as a target for treatment. The term “free” refers to any Hb in the liquid phase of the circulation (such as plasma and serum etc), i.e. outside, but not within, erythrocytes, and therefore also includes protein-bound Hb in the circulation, i.e. not bound in cells; examples of protein-bound Hb is Hb bound to Hp or Hpx. Moreover, the term also encompasses Hb contained in STMBs. In general, the term covers Hb that is not contained in intact erythrocytes. Thus, the term “free Hb” covers all forms of Hb that is not contained in intact erythrocytes.

In this specification, the term “free” as used, inter alia, in the expressions “free Hb”, “free fetal Hb” or “free Hb subunits (e.g. Hbα, Hbβ, Hbδ or Hbγ chains)” refer to Hb, fetal Hb or Hb subunits freely circulating in a biological fluid, as opposed to cellular Hb, which refers to the molecules residing inside cells. The term “free” in this sense is thus mainly used to distinguish free Hb from Hb, which is present in intact erythrocytes. The term does not exclude Hb contained in STMBs and does not exclude Hb bound eg to proteins, but still residing outside the erythrocytes. The same notation applies for HbF, which in the present context relates to free HbF, which in the present context is used to distinguish free HbF from HbF, which is present in intact erythrocytes. The term does not exclude HbF contained in STMBs and does not exclude HbF bound eg to proteins, but still residing outside the erythrocytes.

The terms “marker” or “biomarker”, in this specification, refer to a biomolecule, preferably, a polypeptide or protein, which is differentially present in a sample taken from a pregnant mammal, preferably a woman.

The term “biomarker panel” is used herein for a combination of two or more biomarkers which both must be measured to obtain reliable and reproducible results. Thus, a bo-marker panel for predicting, diagnosing or evaluating the risk for developing PE may be a combination of Hpx and A1M or it may be a combination of Hpx, A1M and free HbF. It is envisaged that further biomarkers, which may be included in such a biomarker panel must be selected from the group consisting of Hp-HbF, Hp, HO-1 and heme.

The term “biological sample from pregnant female mammal”, the term “subject” or equivalents thereof is intended to denote a sample from the maternal side itself; accordingly, the sample is not obtained from e.g. the fetus or the amniotic fluid. The term “sample from the fetus or the fetoplacental circulation” refers to a sample taken from the fetus such as from the amnion fluid, the circulatory system of the fetus including the umbilical cord and the blood vessels within the placenta.

As used herein fetal Hb abbreviated HbF refers to the type of Hb, which is the major component of Hb in the fetus. Fetal Hb has two alpha and two gamma polypeptide chains (Hbα, Hbγ). In the present context, HbF is free circulating HbF, i.e. outside the cells, but it may be bound to other substances such as protein bound to Hp, although not excluding being bound to other proteins.

As used herein alpha-1-microglobulin (A1M), refers to the member of the lipocalin family named alpha-1-microglobulin. Alpha-1-microglobulin may be referred to in literature as A1M, α₁m, HI30, protein HC, and alpha-1-microglycoprotein.

In the following different methods of the invention are discussed. In the individual paragraphs many different details relating eg to nature of samples, reference or control values, sampling time, preferred marker panel etc. are given. The disclosure given under one heading is also relevant for other headings, but are not necessarily repeated. It means that even if there under some of the headings is no mention eg of nature of samples etc., it is clear that the subject covered under the diagnosis aspect also apply in the situations mentioned under the other aspects.

### Biomarker(s) for Preeclampsia and a Method for Diagnosing Preeclampsia

According to the present invention, there is provided a method for the diagnosis or aiding in the diagnosis of PE comprising the following steps:
(a) obtaining a biological sample from a pregnant woman (eg a sample from blood, plasma, urine, cerebrospinal fluid (CSF), placenta biopsies (CVS), uterine fluid and/or amniotic fluid and saliva));
(b) measuring the level of one or more biomarker selected from Hp-HbF, Hp, Hpx, HO-1 and, the level of the biomarker(s) free HbF and/or A1M; or
   measuring the level of Hpx and HO-1;
   or
   measuring the level of one or more biomarker selected from Hp-HbF, Hp, and, the level of the biomarker(s) selected from i) free HbF and/or A1M and/or ii) Hpx and HO-1;

### and Hpx-activity

and
(c) comparing the level of the measured biomarker(s) in the sample with a reference value,
   to determine if said pregnant female has or has not PE, or is or is not at increased risk of developing PE.

More specific, the invention provides a method for the diagnosis or aiding in the diagnosis of PE comprising the following steps:
(a) obtaining a biological sample from a pregnant woman (eg a sample from blood, plasma, serum, urine, cerebrospinal fluid (CSF), placenta biopsies (CVS), uterine fluid and/or amniotic fluid and saliva));
(b) measuring the level of i) Hpx, ii) Hpx and A1M, or iii) Hpx, A1M and free HbF, and optionally one or more of Hp, HO-1 and Hp-HbF
and
(c) comparing the level of the measured biomarker(s) in the sample with a reference value,
   to determine if said pregnant female has or has not PE, or is or is not at increased risk of developing PE.

In some cases, (b) may be expanded to also include A1M and optionally one or more of Hp, HO-1 and Hp-HbF (i.e. without the use of Hpx or free HbF.

As mentioned above, a preferred marker panel according to the present invention and for use in predicting or diagnosing or evaluating the risk for developing PE is: Hpx and A1M optionally supplemented with one or more of the following: free HbF, Hp-HbF, Hp, HO-1.

The control data or reference value is obtained by measuring the level of the above-mentioned markers in pregnant women who do not develop PE. As the level of the individual marker may change dependent on the gestational age, it is preferred that the control or reference value is obtained from pregnant women having the same gestational age (±1 week). As seen from FIG. 11 herein, the normal level—as well as the level indicating a risk for developing PE—changes dependent on the gestational age at which the samples were taken. However, even if such data for reference value should not be available, it is clear from FIG. 11 that the difference between the control level and risk level increases over time, so even if a test sample taken at week 20 is compared with a reference value taken at week 15 the same results should be obtained.

In the methods mentioned herein it is clear that the reference value refers to the actual marker in question.

The sample may be taken at any gestational age. The examples given show that the sample may be taken from week 6 to week 20 or from week 34 to week 40 of gestational age. The advantage of having reliable markers or a panel of marker already at low gestational age is that it is possible early to predict the risk for developing PE and that prophylactic treatment may be instituted to reduce or avoid the symptoms of PE. Moreover, as seen from the examples the marker panel of the invention may enable the evaluation of whether an early or late onset of PE is expected.

The biological sample is preferably a blood sample such as a plasma or serum sample as such samples are most easy to provide.

The invention also collects data for aiding in predicting, diagnosing, evaluating the risk for developing PE or for aiding in evaluating a specific therapeutic or prophylactic treatment of PE.

If desired, the sample can be prepared to enhance detectability of one or more of the biomarkers. Typically, sample preparation involves fractionation of the sample and collection of fractions determined to contain the biomarker(s). Methods of pre-fractioning include, for example, centrifugation, RNA/DNA extraction, size exclusion chromatography, ion exchange chromatography, gel electrophoresis, liquid chromatography, protein fragmentation and protein denaturation.

The step of measuring the level of the biomarker(s) can be accomplished by, for example, an immunological assay (e.g., an ELISA or other solid phase-based immunoassay such as SPRIA or amplified ELISA so called IMRAMP), a protein chip assay, quantitative real-time PCR amplification, surface-enhanced laser desorption/ionization (SELDI), high performance liquid chromatography, Mass Spectrometry, In situ hybridization, immunohistochemistry, chemiluninescence, nephelometry/turbometry, lateral flow or pure or polarized fluorescence or electrophoresis. However, it would be apparent to a person skilled in the art that this list of techniques is not complete and these techniques are not the only suitable methods, which may be used in the present invention for measuring the level of the biomarker(s).

The HbF being detected and/or measured in the methods of the invention include any of the Hb chains (Hbα, Hbβ, Hbδ and Hbγ), or any combination thereof. The gamma chain is indicative of HbF, whereas e.g. the beta and delta chains are indicative of HbA. Based on the disclosure herein, a person skilled in the art will know which Hb chain(s) that should be measured. The Hp molecule consists of two chains, α and β, and two allelic variants of the α-chains exists, α1 and α2. As a result, three phenotypic variants occur in the human population Hp1-1, Hp1-2 and Hp2-2. The term Hp includes all these variants. A1M exists in a free form and in a complex form, bound to other proteins such as IgA, albumin, prothrombin etc. and small molecules and substances, incl. for example heme or radicals. Based on the disclosure herein, a person skilled in the art will know which A1M form(s) that should be measured.

An immunological assay (immunoassay) can, according to the present invention, be used to measure the level of a biomarker. An immunoassay is an assay that uses an antibody to specifically bind an antigen (e.g., Hpx). The immunoassay is characterized by the use of specific binding properties of a particular antibody to isolate, target, and/or quantify the antigen. Thus, under designated immunoassay conditions, the specified antibodies bind to a particular protein at least two times the background and do not substantially bind in a significant amount to other proteins present in the sample. Using the purified markers or their nucleic acid sequences, antibodies that specifically bind to a marker (e.g., Hpx) can be prepared using any suitable methods known in the art [see e.g., Coligan³⁵].

Biomarker level(s) may be measured e.g. using an immunological assay. Particularly, the immunological assay is an ELISA. However, as described above other immunological principles may also be employed.

Free HbF (or another biomarker) level may be determined by measuring free HbF (or another biomarker) RNA. Particularly, free HbF messenger RNA (mRNA) is measured using real-time PCR. In those cases where total Hb level also is determined, this level may also be determined by measuring Hb alpha-chain mRNA, e.g. by using real-time PCR.

Generally, a sample obtained from a subject can be contacted with the antibody that specifically binds the marker. Optionally, the antibody can be fixed to a solid support (however, this does not exclude other non-solid support) to facilitate washing and subsequent isolation of the complex, prior to contacting the antibody with a sample. Examples of solid supports include glass or plastic in the form of, e.g., a microtiter plate, a stick, a bead, or a microbead.

After incubating the sample with antibodies, the mixture is washed and the antibody-marker complex formed can be detected. This can be accomplished by incubating the washed mixture with a detection reagent. This detection reagent may be, e.g., a second antibody which is labelled with a detectable label. Exemplary detectable labels include magnetic beads, fluorescent dyes, radiolabels, enzymes and amplification kits with thyramide (e.g., horse radish peroxidase, alkaline phosphatase and others commonly used in an ELISA), and calorimetric labels such as colloidal gold or colored glass or plastic beads.

Alternatively, the marker in the sample can be detected using an indirect assay, wherein, for example, a second, labelled antibody is used to detect bound marker specific antibody, and/or in a competition or inhibition assay wherein, for example, a monoclonal antibody, which binds to a distinct epitope of the marker is incubated simultaneously with the mixture.

Methods for measuring the amount or presence of an antibody-marker complex include, for example, detection of fluorescence, luminescence, chemiluminescence, absorbance, reflectance, transmittance, refractive index (e.g., surface plasmon resonance, ellipsometry, a resonant mirror method, a gating coupler waveguide method or interferometry) or radioactivity. Optical methods include microscopy (both confocal and non-confocal), imaging methods and non-imaging methods. Electrochemical methods include voltametry and amperometry methods. Radio frequency methods include multipolar resonance spectroscopy.

Useful assays include assay types well known in the art, including, for example, an enzyme immunoassay (EIA) such as enzyme-linked immunosorbent assay (ELISA), radioimmunoassays such as RIA and SPRIA; a Western blot assay; or a slot blot assay but does not exclude other formats that is identified by a person skilled in the art.

The step of measuring the level of biomarker(s) can also be accomplished by detection and measurement of free mRNA coding for Hb polypeptides in the sample, e.g. detection of mRNA sequences coding for the biomarker, or fragments thereof, in the mentioned body fluids.

In the step of comparing the level of a biomarker in the sample with a reference value, the term “reference value” in relation to the present invention refers to a determined baseline or mean level of the biomarker, i.e. the same sort of biomarker being measured in step (b), in samples from a control group. Preferably, the control group comprises pregnant female mammals (preferably women) not diagnosed with or suffering from PE or any other pregnancy related complications, e.g. pregnancy related hypertension.

As the normal level of biomarkers changes with the gestational age of the pregnant woman it is important that the control sample(s) or control value(s) used are representative for the current patient sample analyzed. A control is a sample taken from a pregnant woman who has not or is not at risk of developing PE and is sampled at the similar gestational age (i.e. same number of gestational week). Moreover, the values may depend on the assay applied. Thus, different values may be obtained if an ELISA assay is used compared with values obtained when eg a radioimmunoassay method is applied. A person skilled in the art will find no difficulties in choosing the same analytical method for the test and control sample and will have no difficulties in knowing how to identify the correct gestational age for the test and the control sample.

As seen from the examples herein, reliable and reproducible results are obtained both when the samples are taken at a gestational age of 34-40 weeks and when the samples are taken at a gestational age of 6-20 weeks. Most importantly, the present invention thus provides reliable biomarker(s) that can be used very early in the pregnancy to predict the risk for developing PE and/or to diagnose PE. As seen from Study II reported herein, reliable and reproducible results are obtained when the samples are taken at a gestational age of from between 6 and 20 weeks, notably between 12 and 14 weeks. The results—especially relating to A1M, Hpx and HbF are in accordance with the results reported in Study I, where the samples are taken at 34-40 gestational week. Thus, the present invention provides reliable markers for PE from week 12 (or before) and until birth.

As an example the following values are regarded as normal values when samples are tested at a gestational age of 34-40 weeks and when the assays used for testing Hp, Hpx are ELISA and the assay used for testing non-protein bound A1M is a radioimmunoassay. It should be noted that compared with the normal range given for A1M in a previous patent application WO 2011116958 A1 the values given below are higher, which does not mean that the values given in WO 2011116958 A1 are erroneous, but relate to the fact that A1M in WO 2011116958 A1 and the values reported herein are obtained by the use of two separate radioimmunoassay methods, and are from samples obtained at different gestational ages. This illustrates the importance of using the same method in order to be able to draw a correct conclusion. With respect to HbF there may also be a difference from the values found in WO 2008098734. In the context of Study I, described below, only non-protein bound HbF was measured, whereas in Study II and WO 2008098734 the total HbF (La including the protein-bound part) was measured.

When using a control group, the determination of the reference value of a biomarker is performed using standard methods of analysis well known in the art. The value will of course vary depending on, for example, the type of assay used, the form of the biomarker being measured, kind of biological sample, and group of subjects. For example, normal average plasma levels of a pregnant woman not diagnosed with PE, and measured with an ELISA or radioimmunoassay method as described above and wherein the control samples are taken at a gestational age of 34-40 weeks (the corresponding values when the samples are taken at a gestational age of 12-14 weeks are given in parenthesis), are

i) in the range of from 2 to 5 ng/mL with a median level of 3.85 ng/ml (4.2-7.4 with a median value of 5.6 μg/mL) for free non-protein bound HbF,
ii) in the range of from 0.003 to 1.18 μg/mL with a median level of 0.59 μg/mL for Hp-HbF,
iii) in the range of from 1.04 to 1.30 mg/mL with a median level of 1.17 mg/mL (0.915-1.028 with a median of 0.971 mg/mL) for Hp
iv) in the range of from 0.88 to 0.98 mg/mL with a median level of 0.93 mg/mL (1.111-1.175 with a median of 1.143 mg/mL) for Hpx
v) in the range of from 27.89 to 31.97 μg/mL with a median level of 29.93 μg/mL (14.9-16.1 with a median of 15.5 μg/mL) for A1M,
vi) in the range of from 4.69 to 5.9 ng/mL with a median level of 5.29 ng/mL for HO-1,
vii) in the range of from 52.34 to 67.38 μg/mL with a median level of 59.86 μg/mL for heme.

However, as mentioned above other normal values are expected when the samples are taken at another gestational age. Accordingly, it is preferred to use a gestational-age-correlated control value when comparing values obtained from a test sample with “normal” values.

As illustrated in Study II herein and using the assays described in this study, a pregnant female has or is at increased risk of developing PE if the level of Hpx in a plasma/serum sample from the pregnant female taken at gestational age of 6-20 weeks is 1.1 mg/mL or less, the level of cell-free HbF is 5.6 μg/mL or more and the level of A1M is 15.5 μg/mL or more. When the median values are used, then a pregnant female has or is at increased risk of developing PE if the level of Hpx in a plasma/serum sample from the pregnant female taken at gestational age of 6-20 weeks is 1.06 mg/mL or less, the level of cell-free HbF is 10.8 μg/mL or more and the level of A1M is 17.3 μg/mL or more

In the case were said reference (or normal) value is the level of Hp-HbF, HbF, heme and/or A1M in samples from a control group, a higher level of Hp-HbF, HbF, heme and/or A1M in the sample relative to the reference value indicates that said pregnant female has PE or is at increased risk of developing PE.

In the case where said reference value is the level of Hp, HO-1 and/or Hpx in samples from a control group, a lower level of Hp, HO-1 and/or Hpx in the sample relative to the reference value indicates that said pregnant female has PE or is at increased risk of developing PE.

As seen from the results herein the markers may also be used to determine the risk of developing early or late onset PE. Here especially, the markers Hpx activity and/or free HbF seem to be important. Thus, a combination of a lower Hpx activity with a higher free HbF concentration in a sample—compared to control—is indicative of a late onset, whereas a combination of a higher free HbF concentration with an unchanged Hpx activity is indicative of an early onset PE optionally combined with a higher level of Hp (see Table 3).

The progression (or regression) of the disease can then be followed by frequent measurement of the level of one or more biomarkers in the same type of biological sample of the same pregnant woman.

Another way than looking at the exact plasma level of the biomarker in order to judge whether a pregnant woman is at risk or already has indication of PE, is to look at the standard deviation for the test carried out when determining the plasma/serum level. A relevant parameter is here contemplated to be an increase/decrease from the normal level (e.g. in plasma) with 1.1 times the standard deviation or more. Alternatively, the change in level must be at least 5% from the normal value

The present invention also contemplates the use of the methods described herein in combination with other methods of diagnosis. Diagnostic methods that can be used in combination with the methods of the invention include current methods for diagnosing or aiding in the diagnosing of preeclampsia known to medical practitioners skilled in the art, examples of such methods are described herein before. A biological sample may first be analyzed by the methods described herein. The biological sample may then be tested by other methods to corroborate the observation. Hence, the accuracy of the diagnostic method of the present invention can be improved by combining it with other methods of diagnosis.

As mentioned previously, all details mentioned under the diagnosis aspect also applies for the methods described in other aspects.

### Evaluation of Progression/Regression of Preeclampsia

In further embodiments of the invention, the biomarkers can be employed for determining PE status (e.g. progression or regression). Some of the biomarkers may be used for prognosis, i.e. prediction of the outcome of the disease, of the patient. For example, the concentration of HbF, Hp and/or Hpx correlate with the clinical outcome such as need for NICU treatment, prematurity, and Cesarean section, although not excluding other clinical indications.

Thus, according to an aspect of the present invention, there is provided a method for monitoring the progression or regression of preeclampsia, comprising:
(a) in a first biological sample such as a blood, plasma/serum, urine, CSF, placenta biopsies, uterine fluid or amniotic fluid, isolated from a pregnant female mammal measuring the level of one or more biomarker selected from Hp-HbF, Hp, Hpx, HO-1 and, the level of the biomarker(s) free HbF and/or A1M; or
   measuring the level of Hpx and HO-1; or
   measuring the level of one or more biomarker selected from Hp-HbF, Hp, and, the level of the biomarker(s) selected from i) free HbF and/or A1M and/or ii) Hpx and HO-1;
(b) in a second biological sample such as those mentioned herein, isolated from said pregnant female mammal at a later time measuring the level of the same markers selected under (a) above; and
(c) comparing the values measured in step (a) and (b), wherein
   i) an increase in HbF, Hp-HbF, and/or A1M level(s) in the second sample relative to the HbF, Hp-HbF, and/or A1M level(s) in the first sample, and/or
   ii) a decrease in Hp HO-1 and/or Hpx level(s) in the second sample relative to the Hp, HO-1 and/or Hpx level(s) in the first sample,
   indicates PE progression; and a decrease in i) and/or increase in ii) described above indicates PE regression.

More specifically, the present invention provides a method for monitoring the progression or regression of PE, comprising:
(a) in a first biological sample such as a blood, plasma, urine, CSF, placenta biopsies, uterine fluid or amniotic fluid, isolated from a pregnant female mammal measuring the level of i) Hpx, ii) Hpx and A1M, and/or iii) Hpx, and, optionally, the level of one or more of Hp-HbF, Hp, HO-1
(b) in a second biological sample such as those mentioned herein, isolated from said pregnant female mammal at a later time measuring the level of the same markers selected under (a) above; and
(c) comparing the values measured in step (a) and (b), wherein
   i) an increase in free HbF and/or A1M level(s), and, if measured Hp-HbF, in the second sample relative to the level in the first sample, and/or
   ii) a decrease in Hpx level, and, if measured Hp and/or HO-1 level(s) in the second sample relative to the level in the first sample, indicates PE progression; and a decrease in i) and/or increase in ii) described above indicates PE regression.

In some cases, (a) may be expanded to also include A1M and optionally one or more of Hp, HO-1 and Hp-HbF (i.e. without the use of Hpx or free HbF.

As mentioned above, a preferred marker panel according to the present invention and for use in predicting or diagnosing or evaluating the risk for developing PE is: Hpx and A1M optionally supplemented with one or more of the following: free HbF, Hp-HbF, Hp, HO-1.

It is contemplated that an increase in HbF, Hp-HbF, heme and/or A1M level(s) or a decrease in Hp, HO-1 and/or Hpx level(s) corresponding to 1.1 standard deviations or more is indicative of an increased risk for developing preeclampsia and/or progression of the disease. Alternatively, a variation of 5% from normal values is regarded as an increase (or decrease, if relevant). In an analogous matter a decrease in HbF, Hp-HbF, heme and/or A1M level(s) or an increase in Hp, HO-1 and/or Hpx level(s) corresponding to 1.1 standard deviations or more (or 5% deviation as mentioned above) is indicative of an decreased risk for developing PE and/or regression of the disease.

The details mentioned under the first aspect also apply to this and the following aspects.

### A Method for Assessing the Effectiveness of a Specific Treatment of Preeclampsia

The biomarker(s) and method described above can also be used to assessing the efficacy of a treatment of PE. The only difference being that the first sample is taken either before treatment (denoted time t₀) or during treatment (denoted time t₁), whereas the second sample is taken at a time later than t₀ or t₁, whichever is relevant. The method comprises the following steps:
(a) measuring in a first biological sample isolated from eg blood, plasma or urine of a pregnant female mammal either before or during treatment the level the level of one or more biomarker selected from Hp-HbF, Hp, Hpx, HO-1 and, the level of the biomarker(s) free HbF and/or A1M; or
   measuring the level of Hpx and HO-1; or
   measuring the level of one or more biomarker selected from Hp-HbF, Hp and, the level of the biomarker(s) selected from i) free HbF and/or A1M and/or ii) Hpx and HO-1;
(b) measuring in a second biological sample isolated from eg blood, plasma/serum or urine of said pregnant female mammal at a later time than said first sample the level of one or more biomarker selected under (a);
(c) comparing the values measured in step (a) and (b), wherein
   i) an increase in Hp-HbF, HbF and/or A1M level(s) and/or a decrease in Hp, HO-1 and/or Hpx level(s) in the second sample relative to the Hp-HbF, Hp, HO-1, Hpx, free HbF and/or A1M level(s) in the first sample,
      indicates that the treatment is not effective as PE progresses; and a decrease in Hp-HbF, HbF and/or A1M level(s) and/or an increase in Hp, HO-1 and/or Hpx level(s) described above indicates that the treatment is effective as PE regresses.

More specifically, such a method comprises the following steps:
(a) measuring in a first biological sample isolated from eg blood, plasma/serum or urine of a pregnant female mammal either before or during treatment the level the level of one or more biomarker selected from i) Hpx, ii) Hpx and A1M, and iii) Hpx, A1M and free HbF, and optionally one or more of Hp-HbF, Hp, HO-1
(b) measuring in a second biological sample isolated from eg blood, plasma/serum or urine of said pregnant female mammal at a later time than said first sample the level of one or more biomarker selected under (a);
(c) comparing the values measured in step (a) and (b), wherein
   i) an increase in free HbF and/or A1M level(s), and if relevant Hp-HbF, and/or a decrease in Hpx level and, if relevant Hp, HO-1 level(s) in the second sample relative to the level(s) in the first sample,
      indicates that the treatment is not effective as PE progresses; and a decrease in free HbF and/or A1M level(s), and if relevant Hp-HbF level; and/or an increase in Hpx level and, if relevant, Hp, HO-1 and/or Hpx level(s) described above indicates that the treatment is effective as PE regresses.

In any of the above-methods i)-ix) the marker heme may also be included.

As mentioned above, a preferred marker panel according to the present invention and for use in predicting or diagnosing or evaluating the risk for developing PE is: Hpx and A1M optionally supplemented with one or more of the following: free HbF, Hp-HbF, Hp, HO-1.

In specific embodiments it is contemplated that the efficacy of the treatment can be evaluated by determining whether the decrease or increase corresponds to 1.1 standard deviations or more or a variation of 5% from normal values as described above.

The invention also relates to kits comprising suitable reagents for the determination of the individual markers in a biological sample. Thus, the kits may contain antibodies for the individual markers, means for performing ELISA or any of the other methods mentioned herein.

Substances and Compositions for Use in the Prevention and/or Treatment of Preeclampsia

In accordance with the findings reported herein it is likely that any substance that has i) the ability to inhibit formation of free Hb (free HbF or any other Hb), ii) the ability to bind free Hb (free HbF or any other Hb), or iii) the ability to reduce the concentration of free, circulating free Hb (free HbF or any Hb) to reduce any progression of the disease would be a potential substance for effective treatment and/or prevention of PE. Accordingly, there is provided a use of at least one member selected from the group consisting of Hb binding agents; heme binding/degradation agents: iron-binding agents; agents that stimulate hemoglobin degradation, heme degradation and/or iron sequestering; and/or agents that inhibit placental hematopoiesis for the treatment of PE.

Thus, in one aspect of the invention, in those cases, where a pregnant woman is tested according to the methods has PE or has a risk for developing PE, each of the abovementioned aspects relating to prediction of PE, diagnosis of PE, evaluation of the risk for suffering from PE may be supplemented with a treatment regime involving administration to the pregnant woman one or more of the substances mentioned in the following.

More specifically, it is contemplated that the substance is selected from
i) antibodies or fragments thereof of hemoglobin
ii) haptoglobin
iii) CD 163
iv) alpha-1-microglobulin
v) hemopexin
vi) heme-oxygenase
vii) albumin
viii) transferrin
ix) ferritin

### Hemoglobin-Binders:

### Antibodies

Monoclonal antibodies with strong binding of Hb and blocking of redox enzyme activity of Hb can be developed. The antibodies can be produced by in vivo or in vitro immunization or selected from pre-existing libraries. The antibodies may be selected for specificity against alpha-, beta-delta- or gamma-globin chains, or against common parts of these globin chains. The antibodies can be modified to make them suitable for therapy in humans, i.e. provided with a human immunoglobulin framework. Any part of antibodies may be used: Fv-, Fab-fragments or whole immunoglobulin.

### Haptoglobin

Hp is a glycoprotein found in blood plasma/serum. Three forms of Hp exist, Hp1-1, Hp2-1 and Hp2-2. All forms bind to Hb and forms a Hp-Hb complex. The Hb-Hp complex has weaker redox enzymatic activity than free Hb and does therefore cause less oxidative damage. Binding to Hb prevents, for example, iron loss from the heme group.

### CD163

CD163 is a scavenger receptor, found on macrophages, monocytes and reticuloendothelial system lining the blood vessels. The receptor recognizes the Hp-Hb complex and mediates endocytosis and delivery of this to the lysosomes, degradation by HO-1 (see below) and sequestration of free iron by cellular ferritin. CD163 therefore contributes to the elimination of Hb-induced oxidative stress.

### Heme-Binders/Degraders:

### Hemopexin

Hpx is a glycoprotein (60 kDa) found in human blood plasma/serum, and which eliminates free heme from blood plasma by binding it strongly (Kd appr 1 pmol/L) and transporting the heme to the liver for degradation in the reticuloendothelial system.

### Heme Oxygenase

Heme oxygenase is a cellular heme-binding and degradation enzyme complex that converts heme to biliverdin, carbon monoxide and free iron. The latter is sequestered by cellular ferritin and biliverdin is reduced by biliverdin reductase to bilirubin, which is ultimately excreted into the urine. Three forms of heme oxygenase genes, with very different structures, have been described, HO-1, HO-2 and HO-3. HO-1 is the most important. This gene is upregulated in virtually all cells in the body by Hb, free heme, hypoxia, free radicals, ROS and many different inflammatory signals. HO-1 is a strong anti-oxidant because it eliminates the oxidants heme and iron, but also because it produces bilirubin, which has anti-oxidant effects against some oxidants.

### Albumin

Albumin is a 66 kDa protein in human blood plasma that can bind heme. There is no evidence of cellular uptake and degradation of the albumin-heme complex, and the effect of albumin is probably to act as a depot of heme thus preventing heme from entering endothelial cell membranes, vessel basal membranes, etc.

### Alpha-1-Microglobulin

A1M is synthesized in the liver at a high rate, secreted into the blood stream and transported across the vessel walls to the extravascular compartment of all organs. The protein is also synthesized in other tissues (blood cells, brain, kidney, skin) but at a lower rate. Due to the small size, free A1M is rapidly filtered from blood in the kidneys. A1M has excellent anti-oxidative properties in general and specifically towards oxidative, poisonous degradation products of free Hb; properties that makes it suitable for use in the treatment or prophylaxis of a variety of diseases that involves oxidative stress or wherein the presence of free Hb induces or aggravates a disease or condition.

A1M is an endogenous antioxidant that provides anti-oxidation in several ways. Thus, the present invention relates to A1M, which has been found to combine enzymatic reductase (category 1), non-enzymatic reduction (category 2) and radical-scavenging (category 3) properties. In addition, the non-enzymatic reduction mechanism (category 2) can be employed repeatedly with several cycles of electron-donation. Furthermore, the radical-scavenger mechanism (category 3) result in a net production of electrons that further increases the anti-oxidation capacity of the protein. In other words, the protein carries its own supply of electrons, is independent on cellular metabolism, and can operate both intra- and extracellularly. In addition, A1M can repair oxidative damage that has been inflicted to tissue components (a unique property assigned category 4). See also below for a detailed description of the radical scavenging mechanism.

A1M is a member of the lipocalin superfamily, a group of proteins from animals, plants and bacteria with a conserved three-dimensional structure but very diverse functions. Each lipocalin consists of a 160-190-amino acid chain that is folded into a β-barrel pocket with a hydrophobic interior. Twelve human lipocalin genes are known. Among the human lipocalins, A1M is a 26 kDa plasma and tissue protein that so far has been identified in mammals, birds, fish and frogs. A1M is synthesized in the liver at a high rate, secreted into the blood stream and rapidly (T½=2-3 min) transported across the vessel walls to the extravascular compartment of all organs. The protein is also synthesized in other tissues (blood cells, brain, kidney, skin) but at a lower rate. A1M is found both in a free, monomeric form and as covalent complexes with larger molecules (IgA, albumin, prothrombin) in blood and interstitial tissues. Due to the small size, free A1M is rapidly filtered from blood in the kidneys. The major portion is then readsorbed, but significant amounts are excreted to the urine.

### Sequence and Structural Properties of A1M

The full sequence of human A1M was first reported by Kaumeyer et al. (5). The protein was found to consist of 183 amino acid residues. Since then, at least fifty additional A1M cDNAs and/or proteins have been detected, isolated and/or sequenced from other mammals, birds, amphibians, and fish. The length of the peptide chain of A1M differs slightly among species, due mainly to variations in the C-terminus. Alignment comparisons of the different deduced amino acid sequences show that the percentage of identity varies from approximately 75-80% between rodents or ferungulates and man, down to approximately 45% between fish and mammals. A free cysteine side-chain at position 34 is conserved. This group has been shown to be involved in redox reactions (see below), in complex formation with other plasma proteins and in binding to a yellow-brown chromophore. Computerised 3D models based on the known X-ray crystallographic structures of other lipocalins suggest that Cys34 is solvent exposed and located near the opening of the lipocalin pocket. Complement factor C8γ, another lipocalin, also carries an unpaired Cys in position 34 that is involved in the formation of the active C8 complex.

In the present context the term “alpha-1-microglobulin” intends to cover alpha-1-microglobulin as identified in SEQ ID NO: 1 (human A1M) as well as SEQ ID NO: 2 (human recombinant A1M) as well as homologues, fragments or variants thereof having similar therapeutic activities. Thus, A1M as used herein is intended to mean a protein having at least 80% sequence identity with SEQ ID NO:1 or SEQ ID NO:2. It is preferred that A1M as used herein has at least 90% sequence identity with SEQ ID NO:1 or SEQ ID NO:2. It is even more preferred that A1M as used herein has at least 95% such as 99% or 100% sequence identity with SEQ ID NO:1 or SEQ ID NO:2. In a preferred aspect, the alpha-1-microglobulin is in accordance with SEQ ID NO: 1 or 2 as identified herein. In FIG. 10 is given the sequence listing of the amino acid sequence of human A1M and human recombinant A1M (SEQ ID NOs 1 and 2, respectively) and the corresponding nucleotide sequences (SEQ ID NOs 3 and 4, respectively). However, homologues, variants and fragments of A1M having the important parts of the proteins as identified in the following are also comprised in the term A1M as used herein.

As mentioned above homologues of A1M can also be used in accordance with the description herein. In theory A1M from all species can be used including the most primitive found so far, which is from fish (plaice). A1M is also available in isolated form from human, rat, mouse, rabbit, guinea pig, cow and plaice.

It is important to note that even if A1M and bikunin have the same precursor, they have different amino acid compositions and have different properties. A1M belongs to the so-called lipocalin family whereas bikunin (also denoted ulinastatin) belongs to the protease inhibitor superfamily.

Considering homologues, variants and fragments of A1M, the following has been identified as important parts of the protein for the anti-oxidative effect:

Y22 (Tyrosine, pos 22, basepairs 64-66)

C34 (Cystein, position 34, basepairs 100-102)

K69 (Lysine, pos 69, basepairs 205-207)

K92 (Lysine, pos 92, basepairs 274-276)

K118 (Lysine, pos 118, basepairs 352-354)

K130 (Lysine, pos 130, basepairs 388-390)

Y132 (Tyrosine, pos 132, basepairs 394-396)

L180 (Leucine, pos 180, basepairs 538-540)

1181 (Isoleucine, pos 181, basepairs 541-543)

P182 (Proline, pos 182, basepairs 544-546)

R183 (Arginine, pos 183, basepairs 547-549)

(Numbering of amino acids and nucleotides throughout the document refers to SEQ ID 1 and 3, if other A1M from other species, A1M analogs or recombinant sequences thereof are employed, a person skilled in the art will know how to identify the amino acids of the active site(s) or site(s) responsible for the enzymatic activity.)

Thus, in those cases, where A1M eg has 80% (or 90% or 95%) sequence identity with one of SEQ ID NO: 1 or 2, it is preferred that the amino acids mentioned above are present at the appropriate places in the molecule.

Human A1M is substituted with oligosaccharides in three positions, two sialylated complex-type, probably diantennary carbohydrated linked to Asn17 and Asn96 and one more simple oligosaccharide linked to Thr5. The carbohydrate content of A1M proteins from different species varies greatly, though, ranging from no glycosylation at all in Xenopus leavis over a spectrum of different glycosylation patterns.

A1M is yellow-brown-coloured when purified from plasma or urine. The colour is caused by heterogeneous compounds covalently bound to various amino acid side groups mainly located at the entrance to the pocket. These modifications probably represent the oxidized degradation products of organic oxidants covalently trapped by A1M in vivo, for example heme, kynurenin and tyrosyl radicals (6-8, 10).

A1M is also charge- and size-heterogeneous and more highly brown-coloured A1M-molecules are more negatively charged. The probable explanation for the heterogeneity is that different side-groups are modified to a varying degree with different radicals, and that the modifications alter the net charge of the protein. Covalently linked coloured substances have been localized to Cys34, and Lys92, Lys118 and Lys130, the latter with molecular masses between 100 and 300 Da. The tryptophan metabolite kynurenine was found covalently attached to lysyl residues in A1M from urine of haemodialysis patients and appears to be the source of the brown colour of the protein in this case (6). Oxidized fragments of the synthetic radical ABTS (2,2′-azino-di-(3-ethylbenzothiazoline)-6-sulfonic acid) was bound to the side-chains of Y22 and Y132 (10).

C34 is the reactive center of A1M (9). It becomes very electronegative, meaning that it has a high potential to give away electrons, by the proximity of the positively charged side-chains of K69, K92, K118 and K130, which induce a deprotonization of the C34 thiol group which is a prerequisite of oxidation of the sulphur atom. Preliminary data shows that C34 is one of the most electronegative groups known.

Theoretically, the amino acids that characterize the unique enzymatic and non-enzymatic redox properties of A1M (C34, Y22, K92, K118, K130, Y132, L180, 1181, P182, R183), which will be described in more detail below, can be arranged in a similar three-dimensional configuration on another frame-work, for instance a protein with the same global folding (another lipocalin) or a completely artificial organic or inorganic molecule such as a plastic polymer, a nanoparticle or metal polymer.

Accordingly, homologues, fragments or variants comprising a structure including the reactive center and its surroundings as depicted above, are preferred.

Modifications and changes can be made in the structure of the polypeptides of this disclosure and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids the hydrophilicity values of which are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln₁ His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Trp: Tyr), (Tyr: Trp, Phe), and (Val: Lle, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

In the present context, the homology between two amino acid sequences or between two nucleic acid sequences is described by the parameter “identity”. Alignments of sequences and calculation of homology scores may be done using a full Smith-Waterman alignment, useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Alignment may be made with the FASTA package version v20u6.

Multiple alignments of protein sequences may be made using “ClustalW”. Multiple alignments of DNA sequences may be done using the protein alignment as a template, replacing the amino acids with the corresponding codon from the DNA sequence.

Alternatively different software can be used for aligning amino acid sequences and DNA sequences. The alignment of two amino acid sequences is e.g. determined by using the Needle program from the EMBOSS package (http://emboss.org) version 2.8.0. The Needle program implements the global alignment algorithm described in. The substitution matrix used is BLOSUM62, gap opening penalty is 10, and gap extension penalty is 0.5.

The degree of identity between an amino acid sequence; e.g. SEQ ID NO: 1 and a different amino acid sequence (e.g. SEQ ID NO: 2) is calculated as the number of exact matches in an alignment of the two sequences, divided by the length of the “SEQ ID NO: 1” or the length of the “SEQ ID NO: 2”, whichever is the shortest. The result is expressed in percent identity.

An exact match occurs when the two sequences have identical amino acid residues in the same positions of the overlap.

If relevant, the degree of identity between two nucleotide sequences can be determined by the Wilbur-Lipman method using the LASER-GENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

In a particular embodiment, the percentage of identity of an amino acid sequence of a polypeptide with, or to, amino acids of SEQ ID NO: 1 is determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage. The percentage of identity to, or with, other sequences of the invention is calculated in an analogous way.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution (including conservative and non-conservative substitution), or insertion, and wherein said alterations may occur at the amino- or carboxy-terminus positions of the reference polypeptide sequence or anywhere between those terminal positions, interspersed either individually among the amino acids in the reference sequence, or in one or more contiguous groups within the reference sequence.

Conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. In a second method, translation is carried out in Xenopus oocytes by microinjection of mutated mRNA and chemically aminoacylated suppressor tRNAs. Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. Naturally occurring amino acid residues can be converted to non-naturally occurring species by in vitro chemical modification. Chemical modification can be combined with site-directed mutagenesis to further expand the range of substitutions. Alternative chemical structures providing a 3-dimensional structure sufficient to support the antioxidative properties of A1M may be provided by other technologies e.g. artificial scaffolds, amino-acid substitutions and the like. Furthermore, structures mimicking the active sites of A1M as listed above are contemplated as having the same function as A1M.

### Iron-Binders:

### Transferrin

Transferrin is the most important transporter of iron in blood. The transferrin-iron complex is recognized and bound by cellular receptors, which internalize and dissociate the complex.

### Ferritin

This multimeric protein, consisting of 24 subunits of two types, is the major intracellular depot of free iron. It has a high iron-storing capacity, 4500 iron atoms/ferritin molecule. Bound to ferritin, iron is largely prevented from oxidation and reduction reactions, and hence from causing oxidative damage.

In a further embodiment, the Hb binding agent is an antibody specific for Hb and/or heme.

In specific embodiments, the pharmaceutical preparation comprises a combination of Hb binding agents and/or heme binding agents and/or iron sequestering agents.

Agents that stimulate Hb degradation and/or heme degradation include, but are not limited to, proteins like Hp, Hpx and HO.

A pharmaceutical preparation containing one or more of the substances mentioned above, may be administered to a “placental” animal, such as a human, other primate, or mammalian food animal. A preferred animal for administration is a human or a commercially valuable animal or livestock.

Administration may be performed in different ways depending on what animal to treat, on the condition of the animal in the need of said treatment, and the specific indication to treat. The route of administration may be oral, rectal, parenteral, or through a nasogastric tube provided that the active agent can be transported to the fetal environment such as the fetoplacental circulation, the amnion fluid etc. Parenteral route is preferred. Examples of parenteral routes of administration are intravenous, intraperitoneal, intramuscular, or subcutaneous injection.

Formulation of the pharmaceutical preparation must be selected depending not only on pharmacological properties of the active ingredient but also on its physicochemical properties and the kind administration route. Different methods of formulating pharmaceutical preparations are well known to those skilled in the art.

In general, a pharmaceutical composition comprising A1M (or an analogue, fragment or variant thereof as defined herein) or any of the other substances mentioned herein may be formulated for i.v. administration. Accordingly, the substance can be formulated in a liquid, e.g. in a solution, a dispersion, an emulsion, a suspension etc. As it appears from the examples herein a suitable vehicle for i.v. administration may be composed of 10 mM Tris-HCl, pH 8.0 and 9.125 M NaCl.

For parenteral use suitable solvents include water, alcohols, lipids, vegetable oils, propylene glycol and organic solvents generally approved for such purposes. In general, a person skilled in the art can find guidance in “Remington's Pharmaceutical Science” edited by Gennaro et al. (Mack Publishing Company), in “Handbook of Pharmaceutical Excipients” edited by Rowe et al. (PhP Press) and in official Monographs (e.g. Ph.Eur. or USP) relating to relevant excipients for specific formulation types and to methods for preparing a specific formulation. Suitable excipients include: solvents (e.g. water, aqueous medium, alcohols, vegetable oils, lipids, organic solvents like propylene glycol and the like), osmotic pressure adjusters (e.g. sodium chloride, mannitol and the like), solubilizers, pH adjusting agents, preservatives (if relevant), absorption enhancers, etc.

The substance may be administrated in one or several doses in connection to the radionuclide therapy dose. Preferably, each dose will be administrated i.v. either as a single dose, as a single dose followed by slow infusion during a short time-period up to 60 minutes, or only as a slow infusion during a short time-period up to 60 minutes. Additional doses can be added. When A1M is employed, each dose contains an amount of A1M which is related to the bodyweight of the patient: 1-15 mg A1M/kg of the patient.

For oral compositions, the compositions may be in solid, semi-solid or liquid form. Suitable compositions include solid dosage forms (e.g. tablets including all kinds of tablets, sachets, and capsules), powders, granules, pellets, beads, syrups, mixtures, suspensions, emulsions and the like.

Suitable excipients include e.g. fillers, binders, disintegrants, lubricating agents etc. (for solid dosage forms or compositions in solid form), solvents such as, e.g., water, organic solvents, vegetable oils etc. for liquid or semi-solid forms. Moreover, additives like pH adjusting agents, taste-masking agents, flavours, stabilising agents etc. may be added.

Moreover, specific carriers to target the active substance to a specific part of the body can be included. For example an antibody-A1M complex where the antibody is targeted to placenta (“homing”) by its specificity for a placenta-epitope; a stem cell or a recombinant cell with placenta-homing properties, e.g. integrin-receptors specific for placenta and with the artificial or natural capacity to secrete large amounts of A1M. The treatment would be more efficient since the drug would be concentrated to placenta.

The term “effective amount” in relation to the present invention refers to that amount which provides a therapeutic effect for a given condition and administration regimen. This is a predetermined quantity of active material calculated to produce a desired therapeutic effect in association with the required additives and diluents; i.e., a carrier, or administration vehicle. Further, it is intended to mean an amount sufficient to reduce and most preferably prevent a clinically significant deficit in the activity and response of the host. Alternatively, a therapeutically effective amount is sufficient to cause an improvement in a clinically significant condition in a host. As is appreciated by those skilled in the art, the amount of a compound may vary depending on its specific activity.

Suitable dosage amounts may contain a predetermined quantity of active composition calculated to produce the desired therapeutic effect in association with the required diluents; i.e., carrier, or additive. Further, the dosage to be administered will vary depending on the active principle or principles to be used, the age, weight etc of the patient to be treated but will generally be within the range from 0.001 to 1000 mg/kg body weight/day. Moreover, the dose depends on the administration route.

### LEGENDS TO FIGURES

FIG. 1. Correlation between cell-free HbF- and Hp concentrations—Samples were from normal pregnancies (Control) and women diagnosed with PE. The cell-free HbF plasma concentration of each patient sample (Control and PE) was plotted against the Hp plasma concentration (A). The cell-free HbF plasma concentration of Controls was plotted against the Hp plasma concentration (B). The cell-free HbF plasma concentration of women diagnosed with PE was plotted against the Hp plasma concentration (C). Associations between variables were assessed by linear regression analysis (Pearson's). (Study I)

FIG. 2. Correlation between Hp isoform, cell-free HbF- and Hp-HbF concentration—Hp-isoforms (1-1, 1-2 or 2-2) was investigated in plasma using SDS-PAGE and Western blot with anti-Hp antibodies as shown in the three patient examples (A) as described in Materials and Methods and the distribution of the different isoforms are presented as mean percentage of women with Hp 1-1, 1-2 and 2-2 for respective group (B). The plasma concentration of cell-free HbF (C) and Hp-HbF (D) are shown separately in patient samples with each Hp isoform (Hp 1-1, 1-2 and 2-2). Results are presented as mean percentage of respective Hp isoform (Hp 1-1, 1-2 and 2-2) in B. Results are presented as mean±SEM plasma concentration of cell-free HbF and Hp-HbF in C and D. (Study I)

FIG. 3. Correlation between Hpx concentration and systolic/diastolic blood pressure—Highest systolic (A) and diastolic (B) blood pressure (BP) measured within the last two weeks before delivery were plotted against the plasma concentration of Hpx. (Study I)

FIG. 4. Receiver operating characteristic (ROC) curves—ROC curves showing sensitivity and specificity for the combination of HbF, A1M and Hpx (A), Hpx and A1M (B) and Hpx (C). Area under curve (AUC) is 0.88 for the combination of HbF, A1M and Hpx, 0.92 for the combination of A1M and Hpx and 0.87 for Hpx. (Study I)

FIG. 5. Schematic representation of the tentative chain of events involving HbF, Hp, Hpx, A1114 and ROS and leading to PE—The figure shows a schematic placenta with impaired feto-maternal barrier function causing leakage of placenta factors. 1: Early events in the placenta induce an upregulation of the placenta HbF genes and protein and ROS. 2: Oxidative damage and leakage of the feto-maternal barrier results in 3: increased maternal plasma concentrations of HbF. Excess oxyHb undergoes auto-oxidation reactions resulting in free heme-groups and formation of ROS. 4: A complex network of scavenger proteins, composed of Hp, Hpx and A1M, binds, inhibits and eliminate HbF, heme and ROS. Cell-free HbF is bound by Hp and cleared by CD163 receptor-mediated uptake in monocytes and macrophage-cells. Free heme-groups are bound by Hpx and heme is cleared via the Hpx receptor CD91, preferably expressed on macrophages and hepatocytes. In this study, a highly significant decrease of both the Hp and Hpx was observed in maternal plasma of women with PE as compare to normal pregnancies. This indicated a prolonged presence of increased levels of both extracellular Hb and heme. Analysis of the plasma A1M levels in the present study displayed a significantly increase in women with PE as compared to normal pregnancies, most likely as a result of oxidative stress-induced up-regulation of the A1M gene expression.

FIG. 6. Receiver operating characteristic (ROC) curves—Receiver operation curves of HbF, A1M Hemopexin, Haptoglobin and the combination of these biomarkers as predictive biomarkers of all PE. Specific values are found in Table 10. (Study II)

FIG. 7. Receiver operating characteristic (ROC) curves—Receiver operation curves of the maternal characteristics and the combination of biomarkers and maternal characteristics as markers of all PE. Specific values are found in Table 10. (Study II)

FIG. 8. Correlation between Hpx30 and diastolic blood pressure—Correlation between Hpx30 and diastolic blood pressure in all patients. Specific values are found in Table 5. (Study I)

FIG. 9. Logistic regression analysis—the combination of HbF, Hpx activity, Hpx concentration, heme and HO-1 showed a DR of 84% at 10% 10% false positive rate (FPR) and an AUC of 0.93.

FIG. 10. Sequence listing

FIG. 11. A1M levels compared with control at different gestational age.

### DISCUSSION OF THE RESULTS

In the study the inventors have employed PE as a model disease to study the response of the cell-free Hb-defense network in a pathological situation with prolonged elevation of hemolysis. Thus, in order to investigate the physiological relevance and possible pathophysiological importance of cell-free HbF in the disease progression of PE we investigated the impact of cell-free HbF (both free, denoted HbF, and in complex with Hp, denoted Hp-HbF) on the major human endogenous Hb-scavenging systems: Hp, Hpx, A1M and CD163. This allowed us to investigate the potential for HbF, Hp-HbF, Hb-Total, A1M, Hp, Hpx and CD163 as biochemical markers supporting the diagnosis of PE.

In this study we characterized cell-free HbF and the endogenous Hb- and heme-scavenger systems in pregnant women diagnosed with PE and normal pregnancies (controls) at term. Congruent with previous results, we found a significant increase of HbF in women with PE¹¹. Furthermore, plasma levels of the Hb- and heme scavenger systems were highly affected, displaying significant reduced levels of the Hb-scavenger Hp and the heme-scavenger Hpx. Interestingly, and in line with previously published studies the extravascular heme- and radical scavenger A1M were significantly increased in plasma of women with PE.

In this study we also evaluated the diagnostic and clinical usefulness of the investigated biomarkers and found a clear potential of using these as clinical tools in diagnosing women with PE and or HELLP. Furthermore, the biomarkers displayed a clinical utility, enabling the possibility of identifying women and fetus at risk of clinical complications.

Hemolysis and the subsequent release of cell-free Hb and heme occur in a wide range of clinical conditions and diseases, including HELLP syndrome, transfusion reactions, malaria, hemorrhage, sepsis and sickle cell disease. The release of cell-free Hb and heme causes a range of pathophysiological effects where hemodynamic instability and tissue injury constitutes the major insults. Immediate effects include scavenging of the powerful vasodilator nitric oxide (NO) that leads to increased arterial blood pressure. Furthermore, cell-free Hb and free heme have been described to be accumulated and compartmentalized within the vascular wall and organs, causing subsequent organ failure. Importantly, long-term exposure to cell-free Hb and heme has been described to be associated with NO depletion, inflammation and oxidative stress.

In a series of recent publications the etiological involvement and importance of cell-free HbF and its downstream metabolites free heme and ROS, in the development of PE-related damage and symptoms, have been characterized. Using the dual placenta perfusion system, May et al. showed that addition of cell-free Hb to the fetal circulation caused a significant increase in perfusion pressure, feto-maternal leakage of extracellular Hb into the maternal circulation and morphological damage similar to what is seen in placentas of women with PE. Using a pregnant ewe PE-model and a pregnant rabbit model, it has been shown that starvation causes hemolysis and an increased amount of extracellular heme and bilirubin in the blood. Furthermore, in these models, severe placenta and kidney damage has also been described. This damage was attributed to be caused by the increased hemolysis and subsequent release of Hb and generation of heme and ROS.

Here we report, in line with previous studies, that cell-free HbF (both HbF and Hp-HbF) are significantly increased in pregnant women diagnosed with PE. Thus, these women are presented with an increased blood pressure and protein leakage into the urine, both hallmark of pathophysiological exposure to cell-free Hb and heme.

In order to protect itself against extracellular Hb and free heme, humans have evolved several Hb- and heme-detoxification systems. The most well-investigated Hb-scavenger system is Hp. Hp very efficiently binds extracellular Hb in blood and the resulting Hp-Hb complex is cleared from blood by binding to the macrophage receptor CD163. If Hp becomes depleted as a consequence of large amounts off or prolonged exposure to Hb, excess oxyHb will undergo auto-oxidation reactions resulting in free heme-groups and ROS. Furthermore, excess and non-protein bound Hb will be accumulated within and cause damage to the kidneys, subsequently leading to leakage of proteins into the urine. Depleting, exhausting or overwhelming Hp will allow oxyHb to degrade into its downstream metabolites metHb, free heme and ROS. The major heme-scavenger within the blood stream is Hpx, a highly specific and abundant system that protects cells, vessels and tissue against heme-induces damage. Following binding, Hpx delivers heme via its receptor CD91, preferably expressed on macrophages, hepatocytes, neurons and syncytiotrophoblasts, where it is internalized by receptor-mediated endocytosis and heme is subsequently degraded.

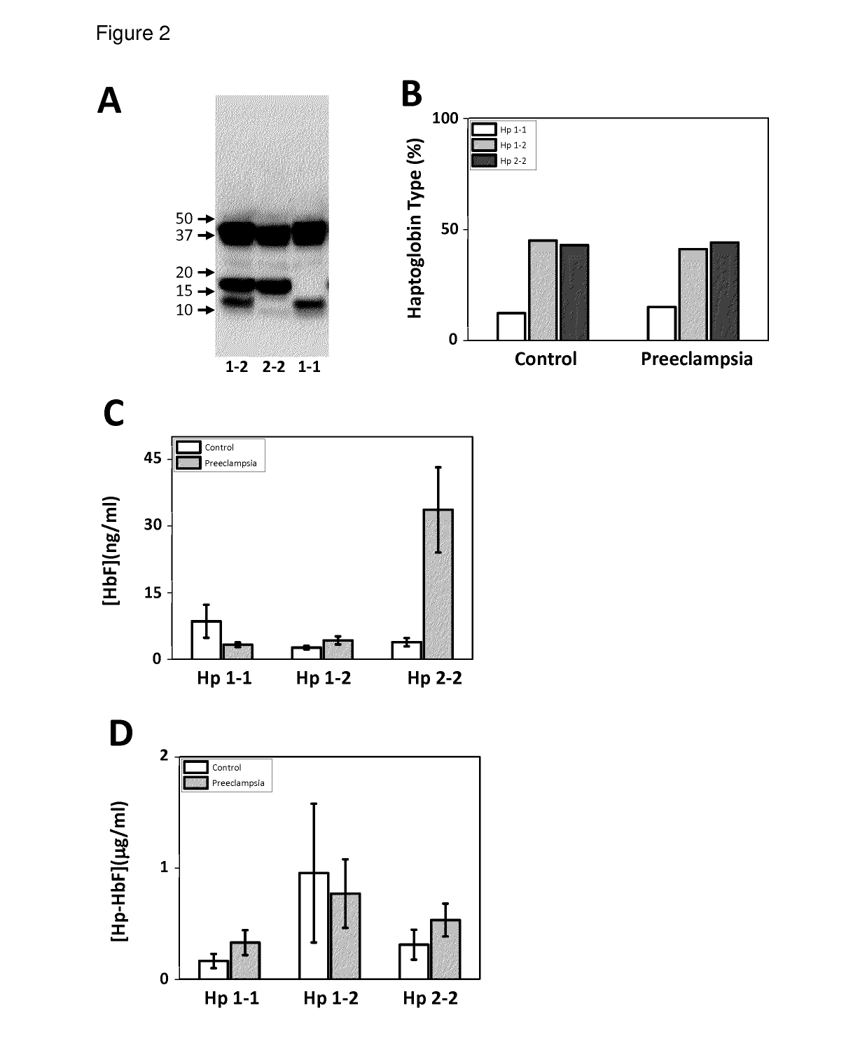

In this study, a highly significant decrease of both the Hp and Hpx were observed in maternal plasma of women with PE as compare to normal pregnancies. This indicated a prolonged presence of increased levels of both extracellular Hb and heme. Thus, although not presented with very high levels of cell-free HbF, we suggest that a continuous exposure to low or moderate level from early pregnancy, e.g. the study by Dolberg et al. suggest an increased level of cell-free HbF as early as gestational week 10-16, will exhaust the endogenous intravascular Hb- and heme (La Hp and Hpx) protective systems. In addition, in some PE patients we observed a highly significant increase in cell-free HbF (non Hp-bound). Very interestingly, all of these patients were found to be of Hp 2-2 isoform (FIG. 2C). Thus, this sub-group of PE patients might have a reduced innate defense against cell-free Hb and in fact might constitute a high-risk patient group.

We have previously shown that the radical scavenger A1M binds and degrades heme and protects cells and tissues from oxidation, damage to mitochondrial-, cellular- and tissue structures and cell death. Furthermore, we have shown that plasma A1M concentration is significantly increased in women with PE both at term and early in pregnancy. Analysis of the plasma A1M levels in the present study confirmed previously published data, displaying a significantly increase of the A1M plasma concentration in women with PE as compared to normal pregnancies.

Why are the A1M-levels increased while the Hp- and Hpx-levels are decreased in the PE patients? It has been shown in several reports that the A1M gene expression is rapidly upregulated in the liver, skin, placenta and other organs as a response to increased levels of Hb, heme and ROS. This will lead to increased secretion of the protein resulting in increased plasma concentrations in pathological situations with increased Hb and ROS loads. Furthermore, no specific receptor-mediated clearance system of A1M has been shown to be triggered during hemolysis or oxidative stress, whereas Hp and Hpx are cleared from plasma upon binding to Hb and heme. As a result, the concentrations of A1M in plasma and extravascular fluids will increase, while Hp and Hpx will be exhausted and hence their plasma concentrations will decrease.

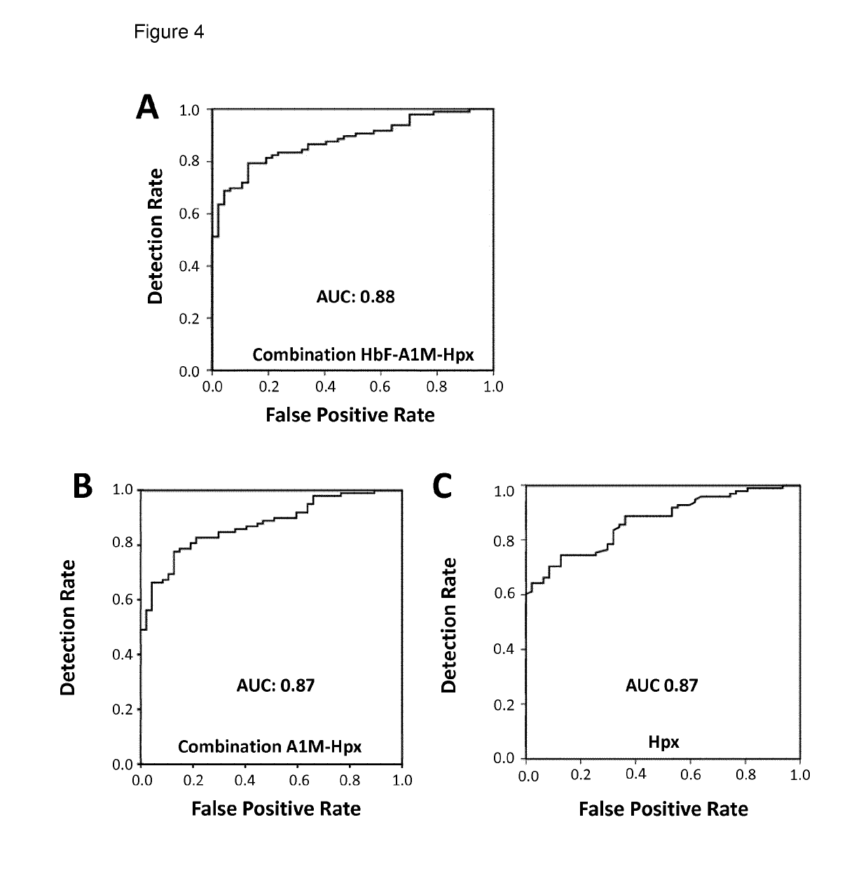

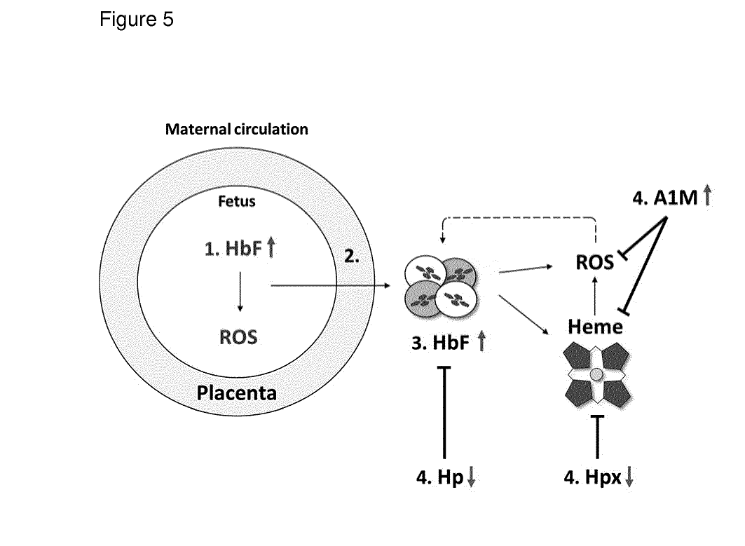

There is an increased attention towards the use of biomarkers in clinical prediction and diagnosis of PE. Several biomarkers have been suggested so far, but no available guidelines recommend the use of biomarkers in a clinical setting. Recently American Congress of Obstetricians and Gynecologists (ACOG) suggested a definition of severe PE where proteinuria is replaced by the use of biomarkers, currently including thrombocytes (<100,000/microliter), serum creatinine (>1.1 mg/dl) and liver transaminases (twice the normal concentration). Here, we present data suggesting that the biomarkers HbF, A1M and Hpx could be used clinically to support the diagnosis of PE. The combination of HbF, Hpx and A1M displayed the highest correlation to diagnosis (detection rate of 69% at 5% false positives, AUC=0.88, FIG. 4A) and the combination of Hpx and A1M also displayed a high detection rate (66% at 5% false positive, AUC=0.87, FIG. 4B). Thus, HbF, Hpx and A1M constitute possible future markers that could support the diagnosis of PE.

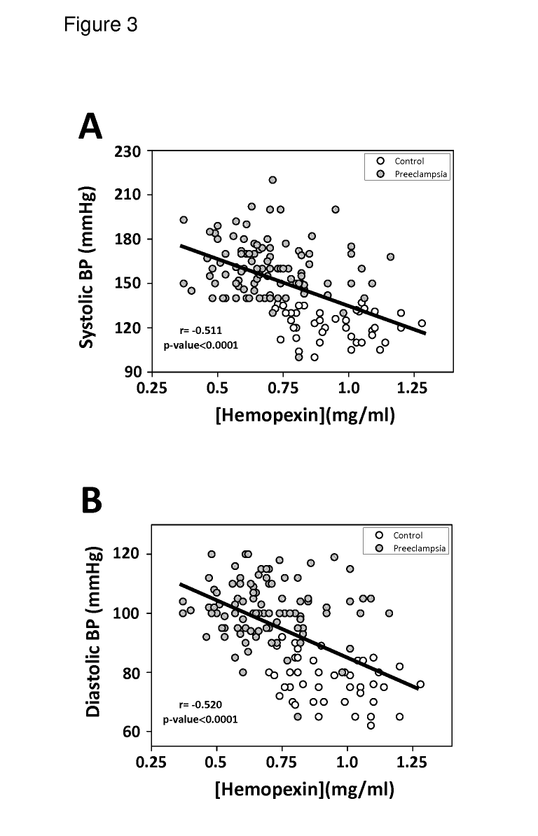

Hpx concentration was shown to have a significant negative correlation to the blood pressure (FIG. 3), i.e. the severity of the disease. Previous studies have shown that active Hpx can affect the renin-angiotensin system (RAS) in in vitro by downregulating the vascular angiotensin II receptor (AT(1)) and promoting an expanded vascular bed^{53,54}. It could be speculated that the increased cell-free HbF levels in women with preeclampsia leads to a consumption of Hpx and consequently a reduced Hpx activity, resulting in an enhanced AT(1) receptor expression and a contracted vascular bed. In fact, Bakker et al⁵⁴ showed that plasma from women with preeclampsia had an increased AT(1) receptor expression on monocytes as compared with plasma from normal pregnancies. This, together with NO consumption, may be important blood pressure regulating effects caused by elevated extracellular HbF observed in PE.

Being able to predict fetal and maternal outcomes is of great clinical value as it can help clinicians in the difficult task to optimize timing of delivery. In this study, the correlation between investigated biomarkers and a range of maternal and fetal outcomes were evaluated. The results indicated that HbF, Hp and Hpx correlated with admission to NICU. Furthermore, Hpx was strongly associated to premature birth. However, since all prematurity in this cohort was associated with preeclampsia this strong association could be as result of the strong correlation between Hpx and preeclampsia rather than prematurity itself.

It is of importance to note that the cohort used in this case-control study is not a normal distributed cohort, i.e. it contains an overrepresentation of women with PE. Consequently, detection and prediction rates reported in this study could therefore be different in a normal distributed cohort, containing 3-8% of PE cases.

In this study, we have among other things characterized cell-free HbF and the endogenous Hb- and heme-scavenger systems in pregnancies complicated by preeclampsia. Plasma levels of HbF were significantly elevated whereas Hp and Hpx were significantly decreased in women with preeclampsia. The extravascular heme- and radical scavenger, and marker of oxidative stress, A1M was significantly increased in preeclampsia plasma. Furthermore, HbF and the related scavenger proteins displayed a potential to be used as clinical biomarkers for more precise diagnosis of preeclampsia and as predictors that help identifying pregnancies with increased risk of obstetrical complications.

In the present study the HO-1 concentration was significantly reduced, particularly in the late onset PE group. The low concentration of HO-1 could be due to continuous strain on this system because of elevated heme and HbF levels throughout PE. The HO-1 enzyme is slowly more and more depleted throughout pregnancy and is therefore lower in late onset PE.

The plasma heme concentration was elevated both in early and late onset PE, however only significantly elevated in late onset PE. The heme concentration obviously correlated well with total Hb concentration. Previously published studies have indicated that the increased levels of HbF throughout the PE pregnancy slowly put a strain on and deplete the maternal Hb and heme scavenging systems including A1M, Haptoglobin and Hemopexin concentration. A constant over-production of HbF in the placenta induces damage to the placenta and the maternal endothelium. The strength of the maternal scavenger and enzyme systems may be important constitutional factors that determine how and when the clinical symptoms present in stage two of PE. The more the systems are strained and/or depleted, the more severe are the clinical symptoms.

Correlation analysis showed a significantly inverse correlation between Hpx activity and diastolic blood pressure in all the patients.

Heme oxygenase 1 was also inversely correlated to systolic and diastolic blood pressure. The higher heme load might explain why HO 1 was lower in PE patients. Depletion of HO-1 diminishes the anti-inflammatory properties, which in turn may aggravate maternal endotheliosis and therefore the blood pressure increases. Furthermore, the degradation of heme by HO-1 produces CO, which is a potent vase-dilator. Diminished levels of HO-1 consequently lead to decreased degradation of heme and less production of CO. This could add to the contracted vascular bed seen in patients with PE.

In this present study, we present a range of potential biomarkers based on HbF and hemoglobin- and heme scavenger proteins and -enzymes. Used in combination, the biomarkers reach a sufficient detection level acceptable for clinical use. The Hpx activity as a single marker was able to detect 30% of PE cases at a 10% FPR. Heme and HO-1 showed similar DRs. Together however, Hpx activity, Hpx, HO-1, Heme and HbF concentrations were able to detect 84% of the PE cases at 10% FPR, which match some of the best biomarkers for PE. Furthermore, several of the biomarkers included in the suggested model correlate with blood pressure and hence with clinical severity of PE.

By measuring components of the Hb metabolism as potential diagnostic biomarkers, a more precise PE diagnosis can be made.

### EXPERIMENTAL

### Materials and Methods

### Study I—Sampling at Gestational Age 34-40 Weeks

### Patients and Demographics

At start, 150 pregnant women were included in the study. The patients were randomly retrospectively selected from a currently on-going prospective cohort study. Exclusion criteria were gestational hypertension, essential hypertension and gestational diabetes. In total 5 cases were excluded due to pre-gestational diabetes or pregnancy related diabetes and a total of 145 patients were included 98 developing PE (cases) and 47 with normal pregnancies (controls). Patient demographics are described in Table 1 and 2.

### Sample Collection

The study was approved by the ethical committee review board for studies on human subjects at Lund University, Sweden. The patients signed informed consent after information given orally and written. Maternal venous sample were taken prior to delivery from patients admitted to the Department of Obstetrics and Gynecology, Lund University Hospital, Sweden. The samples were collected as 6 ml blood into EDTA Vacuette® plasma tubes (Greiner Bio-One GmbH, Kremsmünster, Austria) and centrifuged at 2000×g for 20 minutes. The plasma was then transferred into cryo tubes and stored in −80° C. until time of analysis. Pregnancy outcome for each patient were retrospectively taken from the charts.

Preeclampsia was defined as de novo hypertension after 20 weeks of gestation with 2 readings at least 4 hours apart of blood pressure ≧140/90 mmHg and proteinuria ≧300 mg per 24 hours. This is according to the International Society of the Study of hypertension in Pregnancy's definition⁵⁰. Dipstick analysis was accepted if there was no quantification of proteinuria. Furthermore the PE group was further sub-classified as early-onset PE (diagnosis ≦34+0 weeks of gestation) or late onset PE (diagnosis >34+0 weeks of gestation). There were 3 cases of PE with unknown time of diagnosis and therefore not included in the analyses made with the subgroups of early onset PE and late onset PE.

### Reagents and Proteins

HbF was purified as previously described¹⁶ from whole blood, freshly drawn from umbilical cord blood. Human γ-chains were prepared by dissociation of purified HbF with p-mercuribenzoate (Sigma-Aldrich, St-Louis, Mo., USA) and acidic precipitation as described by Kajita et al.⁵⁵ with modifications by Noble⁵⁶. The absolute purity of HbF (from contamination with HbA) and of γ-chains (from contamination with α- and β-chains) was determined as described previously¹¹. Mouse antibodies to human γ-chains, and hence specific for HbF, were produced and purified by AgriSera AB (Vannas, Sweden). Anti-HbF antibodies were conjugated with horseradish peroxidase (Lightning-Link HRP, Innova Biosciences, Cambridge, UK) as described by the manufacturer. Human A1M was purified from urine as described by Åkerström⁵⁷. Rabbit polyclonal antibodies were prepared against human A1M⁵⁸, mouse monoclonal antibodies against human A1M⁵⁹, goat anti-human A1M and goat anti-rabbit immunoglobulin were prepared as previously described⁶⁰.

### Fetal Hemoglobin (HbF)-Concentrations

A sandwich-ELISA was used for quantification of uncomplexed HbF. Ninety six-well microtiter plates were coated with anti-HbF antibodies (mouse monoclonal, 4 μg/ml in PBS) overnight at room temperature (RT). In the second step, wells were blocked for 2 hours using blocking buffer (1% BSA in PBS), followed by an incubation with HbF calibrator or the patient samples for 2 hours at RT. In the third step, HRP-conjugated anti-HbF antibodies (mouse monoclonal; diluted 1:5000), were added and incubated for 2 hours at RT. Finally, a ready-to-use 3,3′,5,5′-Tetramethylbenzidine (TMB, Life Technologies, Stockholm, Sweden) substrate solution was added. The reaction was stopped after 20 minutes using 1.0 M HCl and the absorbance was read at 450 nm using a Wallac 1420 Multilabel Counter (Perkin Elmer Life Sciences, Waltham, Mass., USA).

### Haptoglobin-Fetal Hemoglobin (Hp-HbF) Concentrations

A sandwich-ELISA was used for quantification of Hp-HbF. This ELISA display a high preference for Hp-HbF compared to uncomplexed HbF (>10× recovery of a Hp-HbF calibrator series compared to a HbF calibrator series at the same molar content of HbF). Ninety six-well microtiter plates were coated with anti-Hp-HbF antibodies (HbF-affinity purified rabbit polyclonal; 4 μg/ml in PBS) overnight at RT. In the second step, wells were blocked for 2 hours using blocking buffer (1% BSA in PBS), followed by an incubation with Hp-HbF calibrator or the patient samples for 2 hours at RT. In the third step, HRP-conjugated anti-Hb antibodies (HbA-affinity purified rabbit polyclonal; diluted 1:5000), were added and incubated for 2 hours at RT. Finally, a ready-to-use TMB (Life Technologies) substrate solution was added, reaction was stopped after 30 minutes using 1.0 M HCl and the absorbance was read at 450 nm using a Wallac 1420 Multilabel Counter (Perkin Elmer Life Sciences).

### Total Hemoglobin (Hb-Total)-Concentrations

The concentrations of Hb-Total in maternal plasma were determined using the Human Hb ELISA Quantification Kit from Genway Biotech Inc. (San Diego, Calif., USA). The analysis was performed according to the instructions from the manufacturer and the absorbance was read at 450 nm using a Wallac 1420 Multilabel Counter.

### Alpha-1-Microglobulin (A1M)-Concentrations

Radiolabelling of A1M with ¹²⁵I (Perkin Elmer Life Sciences) was done using the chloramine T method. Protein-bound iodine was separated from free iodide by gel-chromatography on a Sephadex G-25 column (PD10, GE Healthcare, Stockholm, Sweden). A specific activity of around 0.1-0.2 MBq/μg protein was obtained. Radioimmunoassay (RIA) was performed by mixing goat antiserum against human A1M (diluted 1:6000) with ¹²⁵I-labelled A1M (appr. 0.05 μg/ml) and unknown patient samples or calibrator A1M-concentrations. After incubating overnight at RT, antibody-bound antigen was precipitated by adding bovine serum and 15% polyethylene glycol, centrifuged at 2500 rpm for 40 minutes, after which the ¹²⁵I-activity of the pellets was measured in a Wallac Wizard 1470 gamma counter (Perkin Elmer Life Sciences).

### Haptoglobin (Hp)-Concentrations

The concentrations of Hp in maternal plasma were determined using the Human Hp ELISA Quantification Kit from Genway Biotech Inc. The analysis was performed according to the instructions from the manufacturer and the absorbance was read at 450 nm using a Wallac 1420 Multilabel Counter.

### Hemopexin (Hpx)-Concentrations

The concentrations of Hpx in maternal plasma were determined using the Human Hpx ELISA Kit from Genway Biotech Inc. The analysis was performed according to the instructions from the manufacturer and the absorbance was read at 450 nm using a Wallac 1420 Multilabel Counter.

### Hpx Activity

Plasma Hpx activity was measured in EDTA plasma samples using the Hpx-MCA substrate (synthesized by Pepscan, Lelystad, the Netherlands). The plasma samples (40 μl) were diluted 1:4 with the substrate solution (0.2M Tris+0.9% NaCl pH 7.6 (substrate concentration 80 μM/L) to a final volume of 200 μl. The emission was measured at 460 nm on a Varioskan spectrophotometer (Thermo Fisher) at 37° C. The Hpx activity was measured after 0 min, 30 min (Hpx30), 60 min (Hpx60) and 24 hours. The measured value represented the total amount of serine catabolized by Hpx at the given time point. If the value was <5 after 24 hours of incubation, the activity was considered very low, due to technical problems with either the assay or the samples, and the samples were expelled from further analysis. The area under the curve analysis was based on Hpx30 and Hpx60 measurements (HpxAUC). The measures Hpx30, Hpx60 and HpxAUC mimicked one another and therefore only Hpx30 was used for analysis. In the following Hpx30 is mentioned as Hpx activity.

### Cluster of Differentiation 163 (CD163)-Concentrations

The concentrations of CD163 in maternal plasma were determined using the Human CD163 Duo Set from R&D Systems (Abingdon, UK). The analysis was performed according to the instructions from the manufacturer and the absorbance was read at 450 nm using a Wallac 1420 Multilabel Counter.

### SDS-PAGE and Western Blot

SDS-PAGE was performed using precast 4-20% Mini-Protean TGX gels from Bio-Rad (Hercules, Calif., USA) and run under reducing conditions using molecular weight standard (precision protein plus dual marker Bio-Rad). The separated proteins were transferred to polyvinylidene difluoride (PVDF) or low fluorescence (LF) PVDF membranes (Bio-Rad). The membranes were then incubated with antibodies against Hp (polyclonal rabbit-anti human Hp, 12 μg/ml, DAKO, Glostrup, Denmark). Western blot was performed using HRP-conjugated secondary antibodies (DAKO) and the chemiluminescent substrate Clarity Western ECL (Bio-Rad). The bands were detected in a ChemiDoc XRS unit (Bio-Rad). The relative quantification of A1M bands was performed by densitometry using Image Lab software (Bio-Rad).

### Statistical Analysis

Statistical computer software Statistical Package for the Social Sciences (SPSS Inc., Chicago, Ill.) version 21 for Apple computers (Apple Inc., Cupertino, Calif.) and Origin 9.0 software (OriginLab Corporation, Northampton, Mass., USA) were used to analyze the data.

ANOVA test was used to compare the groups for clinical parameters such as age, BMI, parity, systolic blood pressure, diastolic blood pressure, proteinuria, gestational age at delivery, birth weight, gestational age at time of sampling and APGAR score at 10 minutes.

Mann-Whitney test was used to compare Hpx activities, Hpx, HO-1, heme, HbF and total Hb concentrations between PE and controls. Subgroup-analyses were performed for early- and late onset PE.

The Chi square test was used to compare the groups for fetal gender, labor induction, mode of delivery (e.g. vacuum extraction, caesarean section or vaginal delivery), need of neonatal intensive care unit (NICU) and preterm delivery.

Mean concentration of the examined variables (henceforth referred to as biomarkers) were evaluated in women with PE compared to the control group using non-parametric statistics. A univariate logistic regression model was developed for the evaluated biomarkers. The gestational age at sampling was adjusted for in the logistic regression model. The biomarkers displaying a significant difference were further evaluated using Receiver Operational Curve (ROC-curve) by analyzing the area under the ROC-curve (AUC) as well as calculating the detection rates at different false positive levels. Parallel analysis was performed for each of the examined biomarker as well as different combinations of them. Furthermore, sub-group analysis of women with PE, i.e. early and late onset PE, compared to the control group was performed. The univariate logistic regression model was also used to further calculate fetal outcomes (i.e. admission to NICU and premature delivery and intrauterine growth restriction (IUGR)) and mode of delivery.

### Correlation Analysis

Correlation analysis (Pearson's correlation coefficient) between biomarkers and diastolic- and systolic blood pressure was performed. A p-value of p≦0.05 was considered significant in all tests.

Correlation between Hpx activity and Hpx concentration was calculated using the non-parametric Kendall's correlation coefficient. Furthermore, correlation analysis was performed between Hpx activity and maternal blood pressure (defined as the highest measured blood pressure within 24 hours before delivery).

Correlation analyses were also done between cell-free Hb (HbF and Total Hb), heme, HO-1 and hemopexin concentrations. Furthermore, heme and HO-1 both were correlated to both systolic and diastolic blood pressure.

### Logistic Regression Analysis

The detection rate was determined by ROC-curve analysis for each of the potential biomarkers Hpx, HO-1 and heme. The detection rates were obtained at 10% and 20% false positive rates. The combined detection potential for the biomarkers was obtained by stepwise logistic regression analysis of the biomarkers and ROC-curve analysis.

Results

### Patient Characteristics

The characteristics of the included patients are shown in Table 1 and 2. There was a significant difference between women diagnosed with PE and uncomplicated pregnancies (denoted controls) for age, blood pressure, proteinuria, parity, gestational age at sampling, gestational age of delivery and birth weight. Furthermore, for parameters regarding maternal outcome (e.g. mode of delivery incl. induction and instrumental deliveries) as well as fetal outcome (e.g. admittance to NICU and prematurity) a significant difference was observed. A significant difference in the 10 minutes APGAR score was observed between controls and early onset PE but not late onset PE. There was no significant difference between the groups regarding BMI and fetal gender.

TABLE 1
Patient demographics of PE cases and normal pregnancies (controls). Values
are shown as mean (95% confidence interval) or number (%). Statistical
comparison vs. controls. p-value <0.05 is considered significant.

| | Normal | | Early | Late |
|---|---|---|---|---|
| | pregnancy | | onset | onset |
| | (Control; | Preeclampsia | PE¹ | PE² |
| Outcome | n = 47) | (n = 98) | (n = 22) | (n = 74) |
| Age | 29 | 31** | 32 NS | 30 NS |
| | (28-30) | (30-32) | (30-34) | (29-32) |
| BMI (kg/m²) | 25.0 | 26.1 NS | 27.1 NS | 25.9 NS |
| | (23.7-26.3) | (25.1-27.0) | (24.3-29.9) | (24.9-26.9) |
| Parity (n) | 0.2 | 0.5* | 0.82* | 0.37* |
| | (0.02-0.32) | (0.28-0.64) | (0.23-1.41) | (0.20-0.54) |
| Systolic BP³ (mmHg) | 123 | 161** | 176** | 157** |
| | (120-126) | (157-165) | (167-185) | (153-160) |
| Diastolic BP⁴ (mmHg) | 77 | 101** | 108** | 99** |
| | (75-79) | (99-103) | (103-112) | (97-101) |
| Proteinuria (g/L) | 0.02 | 2.32** | 3.35** | 2.08** |
| | (0.00-0.04) | (2.02-2.61) | (2.68-4.02) | (1.77-2.39) |
| Gestational age at | 282 | 256** | 212** | 269** |
| delivery (days) | (279-285) | (250-262) | (199-225) | (265-273) |
| Twin pregnancies (n) | 0 | 8 (8%) | 2 (9%) | 6 (8%) |
| Gestational age at | 281 | 253** | 208** | 266** |
| sampling (days) | (278-284) | (247-260) | (196-220) | (262-270) |
| Gestational Diabetes⁵ | 0 | 2 (2%) | 0 | 1 (1%)⁶ |
| (n) | | | | |
| Essential | 0 | 3 (3%) | 1 (5%) | 2 (3%) |
| Hypertension⁷ (n) | | | | |
| IVF (n) | 1 (2%) | 8 (8%) | 1 (5%) | 7 (10%) |
| ICSI (=n) | 1 (2%) | 1 (1%) | 1 (5%) | 0 |
| Egg donor recipient (n) | 0 | 1 (1%) | 0 | 1 (1%) |
| Medication to stimulate | 0 | 2 (2%) | 0 | 2 (3%) |
| ovulation⁸ (n) | | | | |

NS: Not significant;
*: p = <0.05;
**: p = <0.001.
¹Early onset PE was defined as diagnosis before 34 + 0 weeks of gestation.
²Late onset PE was defined as gestational week > 34 + 0.
³Highest systolic blood pressure recorded within two weeks prior to delivery.
⁴Highest diastolic blood pressure recorded within two weeks prior to delivery.
⁵Gestational diabetes defined according to Swedish definition; fasting P-glucose ≥7.0 or OGTT with 2 hours P-glucose >12.2 mmol/L.
⁶Time of diagnosis of PE was not known for one patient with gestational diabetes.
⁷Essential hypertension was defined as blood pressure 140/90 before 20 weeks of gestation or condition known before pregnancy.
⁸In one case not known, the other patient medicated with Pergotime.

TABLE 2
Patient demographics of PE cases and normal pregnancies (controls).
Values are shown as mean (95% confidence interval) or number (%).
Statistical comparison vs. controls. p-value <0.05 is
considered significant.

| | Normal | | | |
|---|---|---|---|---|
| | pregnancy | | Early onset | Late onset |
| | (Control; | Preeclampsia | PE¹ | PE² |
| Outcome | n = 47) | (n = 98) | (n = 22) | (n = 74) |
| Birth weight | 3602 | 2834** | 1434** | 3213** |
| (gram) | (3477-3726) | (2621-3047) | (1105-1764) | (3045-3381) |
| Fetal gender | 23:24 | 46:49 NS | 7:15 NS | 37:34 NS |
| (M:F) | | | | |
| HELLP³ | 0 | 7 (7%) | 3 (14%) | 4 (5%) |
| Eclampsia⁴ | 0 | 5 (5%) | 2 (9%) | 3 (4%) |
| Induction (n) | 10 (21%) | 58** (59%) | 2** (9%) | 55** (75%) |
| Vaginal | 35 (75%) | 46* (47%) | 3* (14%) | 43* (59%) |
| delivery (n) | | | | |
| Vacuum | 8 (17%) | 8* (8%) | 0** | 8** (11%) |
| extraction (n) | | | | |
| Cesarean | 12 (26%) | 47** (48%) | 18** (82%) | 27** (37%) |
| section (n) | | | | |
| SGA⁵ | 0 | 1 (1%)^{5a} | 0 | 1 (1%) |
| IUGR⁶ | 0 | 8 (8%) | 5 (23%) | 3 (4%) |
| Admitted to | 2 (4%) | 32** (36%) | 14** (82%) | 18*** (25%) |
| NICU⁷ (n) | | | | |
| Neonatal | 0 | 1 (1%) | 1 (5%) | 0 |
| death | | | | |
| Preterm⁸ (=n) | 0 | 34** (35%) | 20** (95%) | 12** (16%) |
| APGAR10⁹ | 9.80 | 9.75 NS | 9.30* | 9.90 NS |
| | (9.64-9.96) | (9.62-9.89) | (8.80-9.70) | (9.70-10.0) |

NS: Not significant;
*: p = <0.05;
**: p = <0.001.
¹Early onset PE was defined as diagnosis before 34 + 0 weeks of gestation.
²Late onset PE was defined as gestational week >34 + 0.
³HELLP syndrome (Hemolysis, Elevated Liver enzymes, Low Platelets) diagnosed according to Mississippi classification.
⁴Eclampsia was defined as seizures occurring during pregnancy and after delivery in the presence of PE.
⁵SGA (Small for Gestational Age) defined as growth curve on Ultrasonography constant below curve.
^{5a}Patient defined as both SGA and IUGR.
⁶IUGR (Intra Uterine Growth Restriction) was defined as −2 standard deviations (−22%) on Ultrasonography or below 3^{rd} percentile.
⁷NICU (Neonatal Intensive Care Unit).
⁸Preterm was defined as delivery before 36 + 6 weeks of gestation (258 days).
⁹APGAR (Appearance, Pulse, Grimace, Activity, Respiration) score at 10 minutes.

### Cell-Free Hb

The concentration of cell-free HbF, Hp-HbF and Hb-Total were analyzed in all plasma samples from women with PE and controls (Table 3). A 4-fold increase of the HbF concentration was seen in the PE patients (p-value 0.01) as compared to the controls. When subdividing the PE group into early and late onset PE an almost 5-fold increase in the HbF concentration was observed in the early onset PE group as compared to controls (p-value 0.006). In the late onset PE group, an almost 4-fold increase was observed as compared to controls, that was not statistically significant (p-value 0.17). A statistically significant increase in the mean Hp-HbF concentrations was observed for women with PE as compared to controls (p-value 0.018). This difference was not found when comparing early and late onset PE, although a clear trend towards an increase could be seen in the early PE group (p-value 0.15).

No significant difference in Hb-Total concentration was observed between PE vs. controls (p-value 0.53) or between early (p-value 0.80) and late onset PE (p-value 0.73) vs. controls.

TABLE 3
The mean plasma concentrations of the biomarkers in the PE group and
normal pregnancies (controls). Statistical comparison vs. controls.
Significance was calculated with non-parametric statistics
(Mann-Whitney). Values are mean values with
(95% Cl). A p-value <0.05 was considered significant.

| | Normal | | | |
|---|---|---|---|---|
| | pregnancy | | | |
| Bio- | (Control; | Preeclampsia | Early onset | Late onset |
| marker | n = 47) | (n = 98) | PE¹ (n = 2) | PE² (n = 74) |
| HbF | 3.85 | 15.26 | 18.72 | 14.60 |
| (ng/ml) | (2.51-5.20) | (7.0-23.6) | (1.6-39.05) | (5.10-24.0) |
| | | p = 0.01 | p = 0.006 | p = 0.17 |
| Hp-HbF | 0.59 | 0.61 | 1.07 | 0.48 |
| (μg/ml) | (0.003-1.18) | (0.31-0.90) | (−0.10-2.24) | (0.29-0.66) |
| | | p = 0.018 | p = 0.15 | p = 0.02 |
| Total-Hb | 277 | 285 | 290 | 284 |
| (μg/ml) | (232-321) | (238-331) | (152-430) | (237-331) |
| | | p = 0.53 | p = 0.80 | p = 0.73 |
| Hp | 1.17 | 0.97 | 1.34 | 0.89 |
| (mg/ml) | (1.04-1.30) | (0.75-1.19) | (0.39-2.30) | (0.77-1.02) |
| | | p = <0.0001 | p = 0.067 | p = 0.001 |
| CD 163 | 461 | 485 | 433 | 508 |
| (μg/ml) | (408-512) | (445-527) | (324-543) | (465-551) |
| | | p = 0.37 | p = 0.35 | p = 0.07 |
| Hpx | 0.93 | 0.69 | 0.69 | 0.69 |
| (mg/ml) | (0.88-0.98) | (0.66-0.73) | (0.61-0.77) | (0.65-0.73) |
| | | p = <0.0001 | p < 0.0001 | p < 0.0001 |
| Hpx | 0.80 | 0.59 | 0.81 | 0.54 |
| activity | (0.66-0.93) | (0.49-0.69) | (0.54-1.07) | (0.44-0.65) |
| | | p = 0.019 | p = 0.96 | P = 0.004 |
| Heme | 59.86 | 75.03 | 69.54 | 77.55 |
| (μg/ml) | (52.34-67.38) | (67.43-82.62) | (55.07-84.02) | (68.37-86.74) |
| | | p = 0.01 | p = 0.26 | p = 0.02 |
| HO -1 | 5.29 | 4.48 | 4.67 | 4.42 |
| ng/ml | (4.69-5.9) | (4.04-4.93) | (3.37-5.97) | (4.69-5.89) |
| | | p = 0.03 | p = 0.02 | p = 0.01 |
| A1M | 29.93 | 33.50 | 34.07 | 33.70 |
| (μg/ml) | (27.89-31.97) | (31.90-35.10) | (30.31-37.83) | (31.90-35.50) |
| | | p = 0.035 | p = 0.26 | p = 0.03 |

¹Early onset PE was defined as diagnosis before 34 + 0 weeks of gestation.
²Late onset PE was defined as gestational week > 34 + 0

### Hp and CD163

Analysis of the Hp concentration in plasma displayed that the increased HbF concentration in the PE patients was accompanied by a lower Hp concentration (Table 3). The results displayed a highly significant decrease in Hp concentration in plasma samples of women with PE as compared to controls (p-value<0.0001). In addition, late onset PE displayed a significant decrease as compared to the controls (p-value 0.001). In contrast, early onset PE showed a slight but not statistically significant increase in Hp concentration as compared to the controls (p-value 0.067).

Soluble, shedded CD163, the macrophage receptor mediating elimination of the Hp-Hb complex, was analyzed in plasma⁶¹⁻⁶³. The analysis displayed a small but not significant (p-value 0.37) increase in the PE group as compared to the controls (Table 3). Subdividing the PE group into early and late onset PE, a small, not statistically significant, increase was observed in the late onset PE group (p-value 0.07 vs. the controls) whereas a small, not statistically significant, decrease was observed in the early onset PE group (p-value 0.35 vs. the controls).

### Hpx

Analysis of the intravascular heme-scavenger protein Hpx displayed a highly significant decrease in plasma Hpx concentration of women with PE (p-value<0.0001) as compared to the controls (Table 3). Subdividing the PE group, displayed a significant decrease in both the early (p-value<0.0001) and late onset PE (p-value<0.0001) PE groups as compared to the controls.

The blood samples were also analyzed for Hpx activity. Plasma Hpx activity was measured in EDTA plasma samples using the Hpx-MCA substrate (synthesized by Pepscan, Lelystad, the Netherlands). The plasma samples (40 μl) were diluted 1:4 with the substrate solution (0.2M Tris+0.9% NaCl pH 7.6 (substrate concentration 80 μM/L)) to a final volume of 200 μl at 37° C. The emission was measured at 460 nm on a Varioskan spectrophotometer (Thermo Fisher) after 30 min. of incubation (at 37° C.).

Hpx activity was measured spectrophotometrically at following time points: 0 min, 30 min (Hpx30), 60 min (Hpx60) and 24 hours. The measured value represented the total amount of serine sliced by Hpx at the given time point. If the value was <5 after 24 hours the activity was considered extremely low and the samples was expelled from further analysis due to probable damage to the sample. Area under the curve based on Hpx30 and Hpx60 was calculated.

### Hpx Activity

11 of the samples (8 controls and 3 PE) showed “extremely low value” after 24 hours of incubation and were therefore excluded from the analysis.

Hpx activity was significantly lowered in the PE groups compared to the controls group both after 30 min (p=0.02), 60 min (p=0.05) and HpxAUC (p=0.02) (Table 2). However, when dividing the PE patients into early- and late-onset PE it came clear that in the early-onset group Hpx30=0.81 and identical with the control group (Hpx30=0.80) (Table 4). In contradiction to this the late onset group showed an even more markedly decrease in Hpx activity than PE in general concerning all Hpx activities (Hpx30=0.54) (Table 4).

Interesting the ratio between Hpx concentration and Hpx activity can be used to evaluate the risk of developing early or late onset PE. As seen from the table above, the ratio for normal pregnancies is 1.16, whereas it is 0.85 for early onset of PE and 1.28 for late onset of PE. Thus, it the ratio is lower compared to control, i.e. 1 or less then there is an increased risk of developing early onset PE, whereas if the ratio is 1.2 or more there is an increased risk of developing late onset PE, and the Hpx activity is measured as described herein as Hpx30.

### Results for Hpx.

TABLE 4

| | | | Early | Late |
|---|---|---|---|---|
| | Controls | Preeclampsia | onset PE | onset PE |
| | (n = 39) | (n = 96) | (n = 17) | (n = 72) |
| Hpx activity 30 | 0.80 | 0.59 | 0.81 | 0.54 |
| | (0.66-0.93) | (0.49-0.69) | (0.54-1.07) | (0.44-0.65) |
| | | p = 0.019 | p = 0.96 | p = 0.004 |
| Hpx activity 60 | 1.36 | 1.09 | 1.33 | 1.04 |
| | (1.09-1.62) | (0.97-1.22) | (1.06-1.61) | (0.89-1.19) |
| | | p = 0.046 | p = 0.92 | p = 0.02 |
| Hpx activity | 0.74 | 0.57 | 0.74 | 0.53 |
| AUC | (0.61-0.87) | (0.49-0.65) | (0.55-0.93) | (0.44-0.62) |
| | | p = 0.022 | p = 0.99 | p = 0.007 |
| Hpx plasma | 0.93 | 0.69 | 0.69 | 0.69 |
| concentration¹ | (0.88-0.98) | (0.66-0.73) | (0.61-0.77) | (0.56-0.73) |
| | | p < 0.0001 | p < 0.0001 | p < 0.0001 |

¹Previously mentioned herein

### Correlation Analysis.

Hpx activity was not correlated to Hpx plasma concentration (p=0.74 for Hpx30). This was neither the case in the early onset group (p=0.17) nor the late onset PE group (p=0.24).

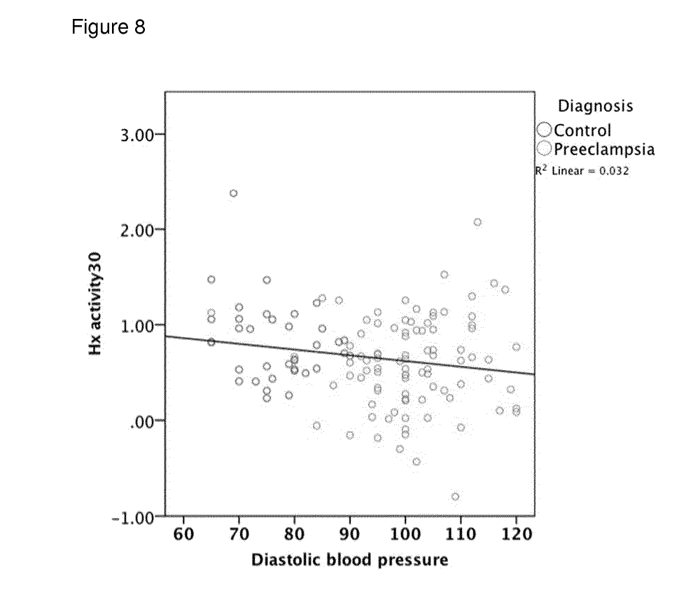

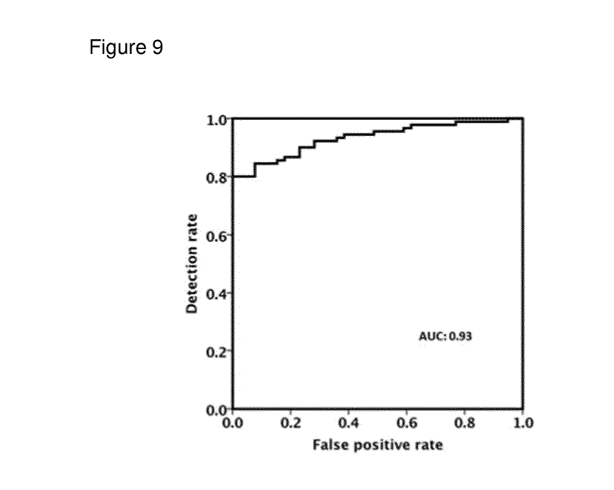

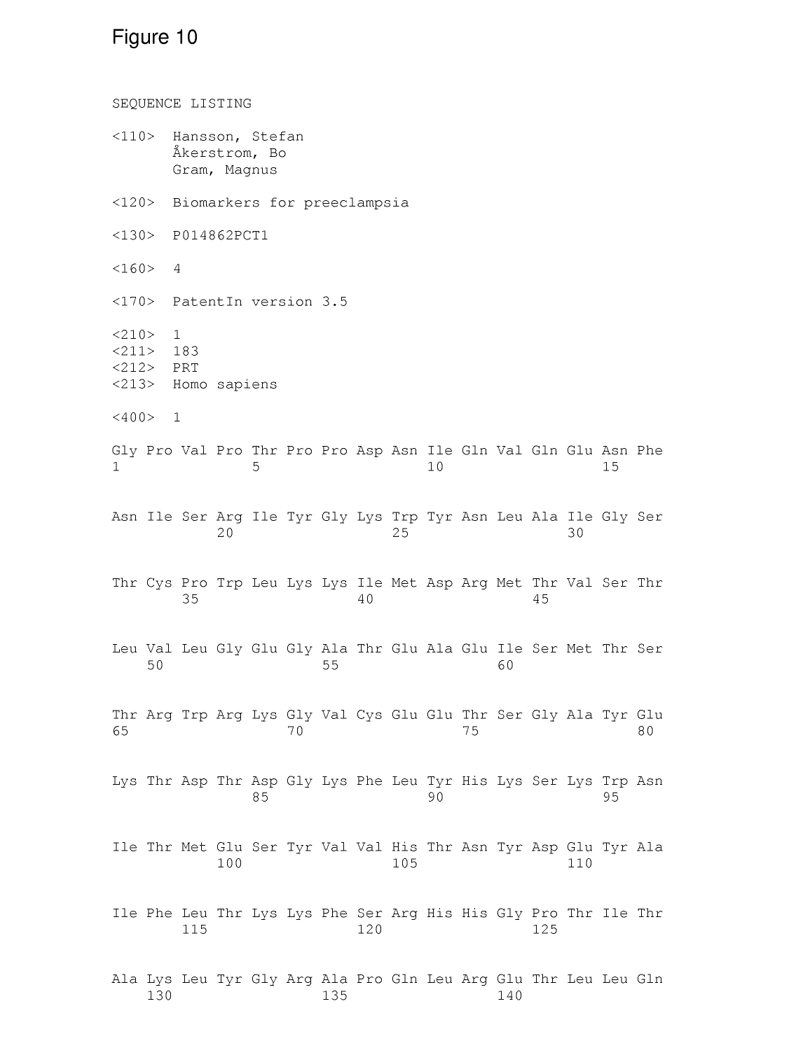

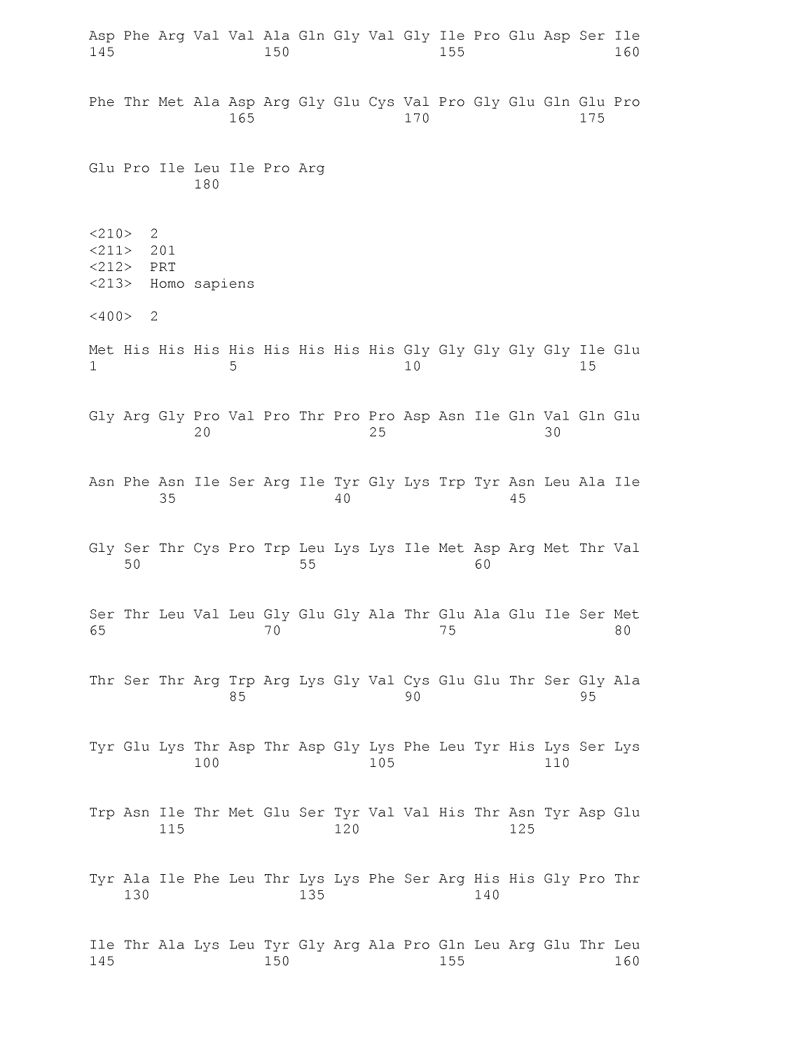

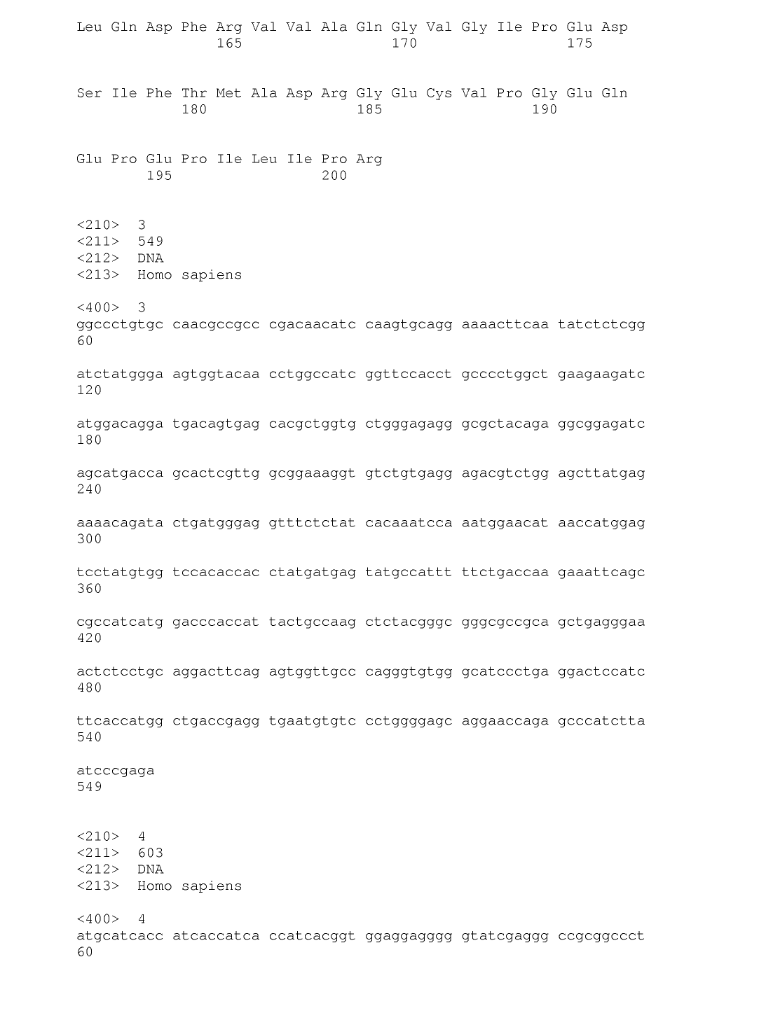

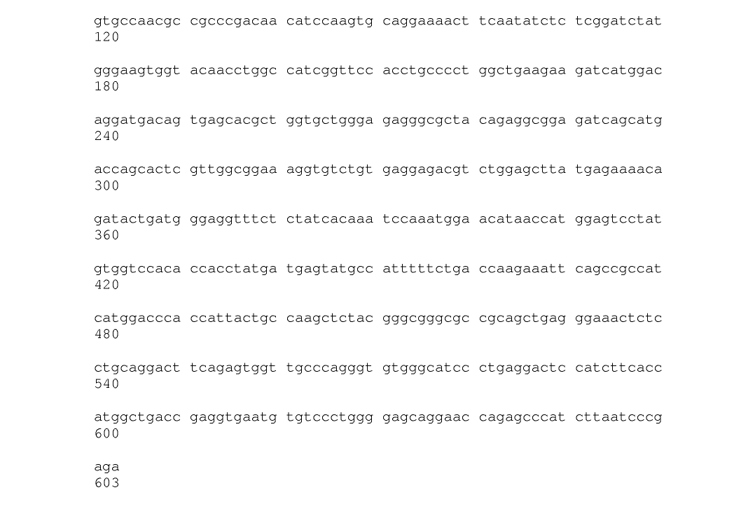

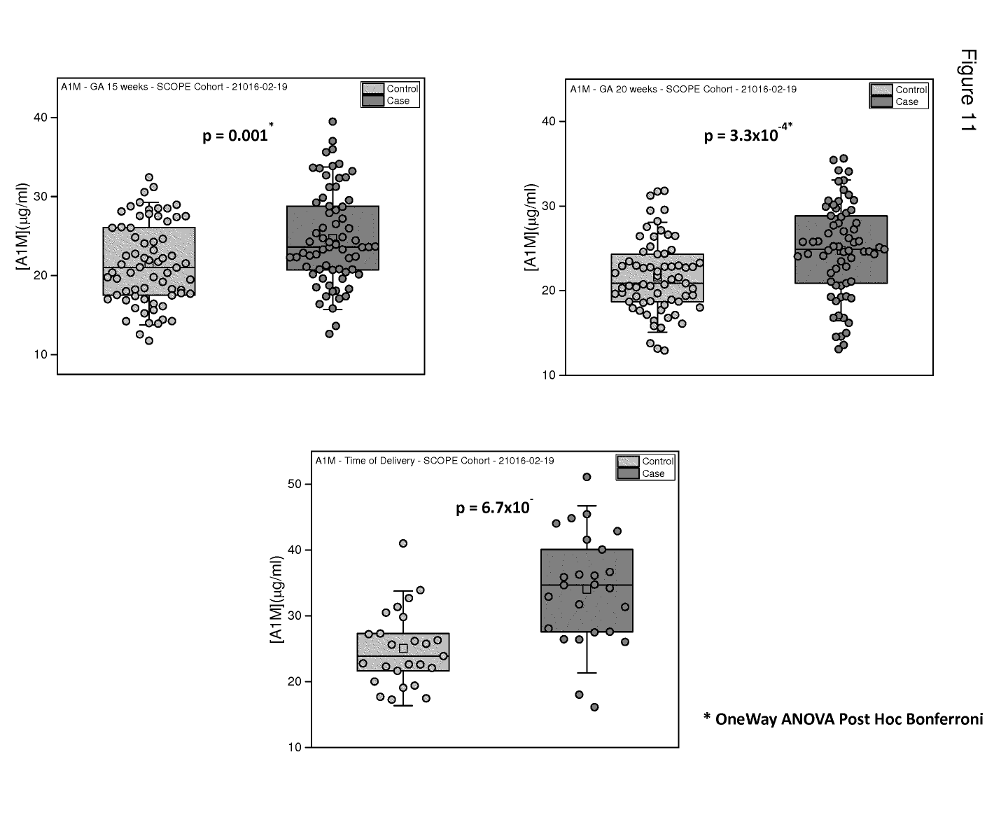

Hpx30 was significantly correlated to diastolic blood pressure in all patients (p=0.04) and there was a clear tendency towards correlation for Hpx60 (p=0.1) and HpxAUC (p=0.06) (Table 4). When the early-onset patients were expelled from the analysis there was a clear correlation between diastolic blood pressures and each of Hpx30, Hpx60 and HpxAUC (Table 5, FIG. 8). Furthermore there were clear tendencies towards correlation between systolic blood pressure and Hpx30 (p=0.07), Hpx60 (p=0.17) and HpxAUC (p=0.11) (Table 5).

### Results for Blood Pressure:

TABLE 5

| | Hpx30 | Hpx60 | HpxAUC |
|---|---|---|---|
| All patients | | | |
| Systolic blood | p = 0.35 NS | p = 0.53 NS | p = 0.45 NS |
| pressure | | | |
| Diastolic blood | CF = −0.18 | p = 0.10 NS | CF = −0.17 |
| pressure | p = 0.04 | | p = 0.06 NS |
| Late onset PE | | | |
| and controls | | | |
| Systolic blood | CF = −0.17 | p = 0.17 NS | p = 0.11 NS |
| pressure | p = 0.07 | | |
| Diastolic blood | CF = −0.25 | CF = −0.20 | CF = −0.23 |
| pressure | p = 0.009 | p = 0.04 | p = 0.02 |

CF: Pearson's correlation factor.

In concordance to previous findings we found decreased Hpx activity in patients with manifest PE. However we did only find Hpx activity to be decreased in patients with late-onset PE but not in early-onset PE. Contrary to this and as described herein, Hpx protein concentration has been shown to be statistically significantly decreased in both early and late onset PE. Correlation analysis showed statistically significant inverse correlation between Hpx30 and diastolic blood pressure in all the patients and there was a tendency towards the same inverse correlation for Hpx60 and HpxAUC (Table 5). When only analyzing the correlation in the controls and late onset groups together there was statistically significant correlation between all of Hpx-activities and diastolic blood pressure and a tendency towards statistically significant correlation to systolic blood pressure.

### A1M

Analysis of plasma levels of the heme- and radical scavenger A1M displayed a significant increase of plasma A1M concentration in women with PE (p-value 0.035) as compared to controls (Table 3). Subdividing the PE group, a statistically significant increase was observed in the late onset PE group (p-value 0.03) and a clear, but not statistically significant, increase was seen in the early onset PE group (p-value 0.26).

### Correlation Cell-Free HbF and Hp

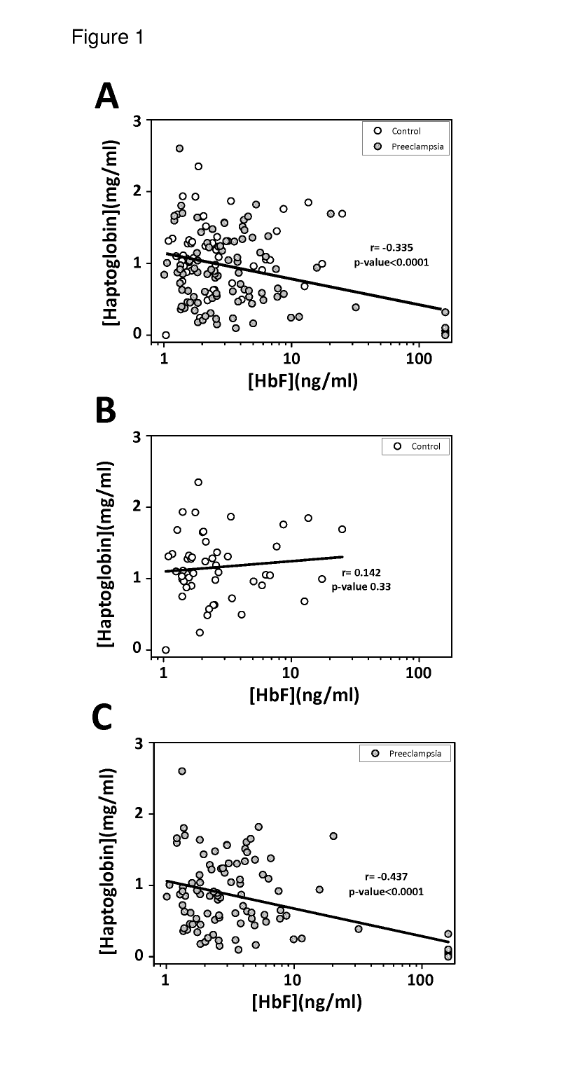

The correlation between plasma cell-free HbF and Hp levels was evaluated. A negative correlation was found, i.e. an increased plasma cell-free HbF concentration was associated with a decreased plasma Hp concentration, when including all patients, controls and women with PE (r=−0.335, p-value<0.0001, n=145)(FIG. 1A). Strikingly, when comparing the correlation in controls (FIG. 1B) and women with PE (FIG. 1C) separately, an increased negative correlation was observed for the PE group (r=−0.437, p-value<0.0001, n=98) whilst in the control group a weakly positive correlation was observed (r=0.142, p-value 0.33, n=47). Similar correlations was observed for Hp vs. Hp-HbF and Hp vs. Hb-Total, but none of them reached statistical significance (Hp vs. Hp-HbF r=−0.05, p-value 0.52; Hp vs. Hb-Total r=0.03, p-value 0.73).

### Association Between Hp Isoforms and Level of Cell-Free HbF, Hpx and A1M

We identified the predominant Hp-isoform (1-1, 1-2 or 2-2) in the patient plasma samples using Western blot (FIG. 2A). As seen in FIG. 2B, a similar distribution of the different isoforms were observed in both controls and PE, with a predominant presence of Hp 1-2 (C, 45%; PE, 41%) and 2-2 (C, 43%; PE, 44%) as compared to 1-1 (C, 12%; 15%). Subdividing the PE group in to early and late onset PE also displayed a similar distribution 1-1 (early, 13%; late, 15%), 1-2 (early, 45%; late, 40%) and 2-2 (early, 42%; late, 45%). Furthermore, the association between the Hp-isoforms and the plasma levels of cell-free HbF, Hp-HbF, Hb-Total, Hp, CD163, Hpx and A1M were analyzed (FIG. 2C-D). A striking increase in the concentration of cell-free HbF was observed in the Hp 2-2 group of women with PE (FIG. 2C). A smaller, but similar increase in the concentration of Hp-HbF was observed in the Hp 2-2 PE group as compared to controls (FIG. 2D). No additional significant associations with the Hp isoform were observed.

### Correlation Analysis Between Biomarkers and Disease Severity

Correlation analysis using Pearson's correlation coefficient showed highly significant inverse correlation between Hpx and blood pressure, both systolic (r=−0.511, p-value<0.00001, n=145) and diastolic (r=−0.520, p-value<0.00001, n=145)(FIG. 3). No statistical significant correlation was observed for any of the other biomarkers and blood pressure.

### Evaluation of Biomarkers as Diagnostic Tools and Clinical Predictors

A logistic regression models was used to evaluate the usefulness of the described biomarkers as diagnostic markers of PE. Comparing women with PE vs. controls, a significant difference was detected for HbF, A1M and Hpx (p-value<0.0001) but not for Hp and CD163. Each of the significantly altered biomarkers were able to diagnose PE (adjusted for gestational age) but Hpx showed the high level of significance and a diagnostic detection rate of 64% at a false positive rate of 5% with an AUC of 0.87 (Table 6, FIG. 4C). The combination of Hpx, A1M and HbF was not significant (p-value for HbF 0.08) but displayed a diagnostic detection rate of 69% at a false positive rate of 5% with an AUC of 0.88 (Table 6, FIG. 4A). The combination Hpx and A1M was significant and showed a diagnostic detection rate of 66% at a false positive rate of 5% and an AUC of 0.87 (Table 6, FIG. 4B).

TABLE 6
Sensitivity and specificity values for the combination of 1) HbF,
A1M and Hpx, 2) A1M and Hpx and 3) Hpx alone. Detection
rates for PE at different false positive rates and AUC for the ROC
curve. Calculations are for all PE vs. controls.

| False positive | HbF combined with | A1M combined | |
|---|---|---|---|
| rate | A1M and Hpx¹ | with Hpx² | Hpx |
| 5% | 69% | 66% | 64% |
| 10% | 69% | 67% | 70% |
| 20% | 81% | 81% | 75% |
| 30% | 83% | 85% | 79% |
| AUC | 0.88 | 0.87 | 0.87 |

¹Based on logistic regression including all three parameters.
²Based on logistic regression including both parameters.

### Prediction of Fetal and Maternal Outcomes

Beside the test of the biomarkers to support diagnosis of PE we examined whether the biomarkers could predict a range of fetal and maternal outcomes. This was done with a logistic regression model similar to the model of PE. The tested fetal outcomes were: admission to NICU, IUGR and prematurity. The tested maternal outcomes were: induction of labor, cesarean section and vacuum extraction. The biomarkers HbF (p-value 0.001), Hpx (p-value 0.008) and Hp (p-value 0.03) each showed potential as predictive biomarkers of “admission to NICU”. However, in a combined logistic regression model they turned out insignificant. The biomarkers Hpx (p-value 0.0003, AUC=0.71) and CD163 (p-value 0.03, AUC=0.61) showed potential as biomarkers of prematurity. In combination these two biomarkers proved significant with a slightly stronger association to prematurity (p-value 0.001 and p-value 0.025, AUC 0.72).

None of the biomarkers showed any predictive value concerning induction of labor or vacuum extraction. Hpx displayed a significant association with Cesarean section (p-value 0.009, AUC 0.62).

TABLE 7
Area under the ROC-curves (AUC) for fetal outcomes (admittance to
Neonatal Intensive Care Unit (NICU) and prematurity) and maternal
outcomes (risk of cesarean section). The fetal outcome and the maternal
outcomes induction of labor and vacuum extraction were not significantly
related to any of the biomarkers. All calculations were based on
univariable logistic regression analysis.

| | Significance | AUC |
|---|---|---|
| Admittance to NICU | | |
| HbF | 0.001 | 0.69 |
| Hp | 0.03 | 0.62 |
| Hpx | 0.008 | 0.66 |
| Prematurity | | |
| Hpx | 0.001 | 0.70 |
| CD 163 | 0.04 | 0.61 |
| Combination | 0.001 | 0.72 |
| Hpx + CD 163 | 0.025 | |
| Cesarean section | | |
| Hpx | 0.009 | 0.62 |

### Study II—Sampling at Gestational Week 6-20

### Patients and Samples

The study was approved by the ethical committees at St Georges University Hospital, London, UK. All participants signed a written informed consent prior to inclusion. Women attending a routine antenatal care visit at St. Georges Hospital Obstetric Unit, London were recruited during the years 2006 and 2007.

Gestational length was calculated from the last menstrual period and confirmed by ultrasound crown-rump-length measurement. A maternal venous blood sample was collected at 6-20 weeks of gestation (mean 13.7) in a 5 ml vacutainer tube (Becton Dickinson, Franklin Lakes, N.J.) without additives. After clotting, the samples were centrifuged at 2000×g at room temperature for 10 minutes and serum was separated and stored at −80° C. until further analysis.

All pregnancy outcome-data was obtained from the main delivery suite database and checked for each individual patient. PE was defined as in Study I herein.

As in Study I, normal pregnancy was defined as delivery at or after 37+0 weeks of gestation with normal blood pressure. The uncomplicated pregnancy (control) samples were recruited as consecutive cases during the same time period.

### Measurement of Total Hb, HbF, A1M, Hp and Hpx

HbF-concentration in serum samples (La cell-free HbF) was measured with a sandwich ELISA using polyclonal antibodies as described in Study I. The A1M concentration was determined by a radioimmunoassay as described in Study I. Hb-Total, Hp and Hpx concentration were serum samples using ELISA Quantification Kit for respective marker as described in Study I.

### Statistical Analysis

SPSS statistics version 21.0 for Apple computers was used along with the statistical software R studio Version (0.98.1062). A p value ≦0.05 was considered significant in all analyses. Significant differences between the groups for the biomarkers HbF, Hb-Total, Hp, Hpx, and A1M were calculated with one-way ANOVA. Due to differences in gestational age when Doppler ultrasound was performed in the PE and control groups, UtADs were transformed into Multiples of the Median (MoM)-values according to mean values given by Velauthar et al⁶⁴.

Stepwise regression analysis is a commonly used method for developing prediction models but has been criticized⁶⁵. We therefore attempted to validate the results also by developing prediction models by two more recently developed statistical methods, Lasso regression and boosted tree regression and compare these methods in terms of their prediction capability. The methods were compared by area under the ROC-curve. In order to validate the prediction results the dataset was randomized into a training cohort (⅔) which was used for developing the prediction models and a test cohort (⅓) used for testing their predictive ability.

The final models of the biomarkers and maternal characteristics were built on backwards stepwise logistic regression. Separate analyses were performed for early onset PE and late onset PE. For all regression parameters and the parameters in combination ROC-curves were performed and the prediction rates (PR) at different false positive rates (FPR) were calculated. The optimal prediction rate/FPR was defined as the point in the ROC-curve closest to the upper left corner.

### Results

### Demographics

In total, 520 women were included, out of which 86 developed PE (cases), 65 had spontaneous preterm birth (SPTB), 7 were complicated by IUGR, 10 developed pregnancy induced hypertension (PIH), 1 patient had IUGR and placental abruption, 3 had isolated placental abruption (without PE or IUGR), 2 had essential hypertension. 347 women with uncomplicated pregnancies and term delivery (>37 weeks of gestation) were included as controls.

The maternal characteristics are shown in Table 8. Of the 86 women who developed PE, 28 were delivered before 37+0 weeks of gestation. Out of these, 17 were delivered before 34+0 weeks of gestation showing a significantly lower birth weight compared to controls. The groups essential hypertension without PE (n=2) and abruption (n=3) were excluded from the following analysis due to small sample size.

| | | | | Pregnancy | Essential hypertension | Spontaneous | |
|---|---|---|---|---|---|---|---|
| | Control | Preeclampsia | IUGR | induced hypertension | (without PE) | preterm birth | Abruption |
| | (n = 347) | (n = 86) | (n = 7) | (n = 10) | (n = 2) | )n = 64) | (n = 3) |
| Ethnic origin | | | | | | | |
| Caucasian (304) | 252 | 41 | 4 | 6 | 2 | 34 | 1 |
| South Asian (70) | 36 | 18 | 2 | 1 | 0 | 13 | 0 |
| Black (54) | 20 | 21 | 0 | 3 | 0 | 10 | 0 |
| East Asian (4) | 3 | 0 | 0 | 0 | 0 | 1 | 0 |
| Mixed (19) | 13 | 2 | 0 | 0 | 0 | 4 | 0 |
| Not known (38) | 23 | 4 | 1 | 0 | 0 | 3 | 2 |
| | | p < 0.000001* | p = 0.51 NS | 0.06 NS | | p = 0.004* | |
| Gravidae | 1.46 | 2.73 | 3.33 | 3.44 | 1.0 | 2.71 | 4.67 |
| | (1.36-1.56) | (2.32-3.14) | (−1.15-7-82) | (0.03-6.86) | (1.0-1.0) | (2.2-3.21) | (−3.32-12.65) |
| | | p < 0.0001 | p < 0.0001 | p < 0.0001 | p = 0.49 NS | p < 0.0001 | p < 0.0001 |
| Para | 0.11 | 1.14 | 0.67 | 2.11 | 0.0 | 0.74 | 0.67 |
| (Mean-95% CI) | (0.06-0.16) | (0.78-1.14) | (−0.19-1.52) | (−0.85-5.07) | 1.0 (0.0-0.0) | (0.45-1.03) | (−2.2-3.5) |
| | | p < 0.0001 | P = 0.003 | p < 0.0001 | p = 0.73 NS | p < 0.0001 | p = 0.04 |
| Body Mass Index | 23.4 | 26.9 | 27.4 | 28.2 | 20.5 | 23.5 | 21.1 |
| | (22.9-23.9) | (25.5-28.35) | (20.9-33.8) | (21.7-34.8) | (3.3-37.6) | (21.9-25.1) | (16.2-25.9) |
| | | p < 0.0001 | p = 0.02 | P = 0.001 | p = 0.36 NS | p = 0.88 NS | p = 0.37 NS |
| GA at ultrasound | 12.5 | 18.5 | 20.4 | 16.3 | 11.6 | 20.4 | 19.0 |
| scanning | (12.4-12.6) | (17.5-19.5) | (15.9-25.0) | (12.4-20.3) | (7.9-15.2) | (19.5-21.4) | (5.8-32.3) |
| (Mean-95% CI) | | p < 0.0001 | p < 0.0001 | p < 0.0001 | p = 0.10 NS | p < 0.0001 | p < 0.0001 |
| GA at blood | 13.5 | 13.9 | 13.6 | 13.8 | 12.0 | 14.1 | 13.2 |
| sampling (Mean- | (13.3-13.8) | (13.3-14.6) | (10.5-16.7) | (12.3-15.4) | (−24.3-48.3) | (13.4-14.8) | (7.9-18.6) |
| 95% CI) | | p = 0.17 NS | p = 0.94 NS | P = 0.66 NS | 0.34 NS | p = 0.09 NS | p = 0.83 |
| Fetal gender | | | | | | | |
| Male | 185 | 49 | 2 | 4 | 1 | 32 | 2 |
| Female | 161 | 36 | 4 | 6 | 1 | 32 | 1 |
| | | p = 0.44 NS* | p < 0.0001 * | p = 0.69 NS* | NS* | p = 0.8 NS* | NS* |
| Birth weight | 3467 | 2716 | 1791 | 2810 | 3260 | 2324 | 1838 |
| | (3415-3520) | (2485-2947) | (1214-2369) | (2090-3531) | (3414-3518) | (2160-2488) | (47-3629) |
| | | p < 0.0001 | p < 0.0001 | p < 0.0001 | p = 0.56 NS | p < 0.0001 | p < 0.0001 |
| Prematurity (%) | 0% | 28 (33%) | 7 (100%) | 4 (40%) | 0 (0%) | 60 (100%) | 2 (100%) |
| | | p < 0.0001 | p < 0.0001 | p < 0.0001 | | | |
| Mean GA at delivery | 40.4 | 36.7 | 34.8 | 37.0 | 39.4 | 34.6 | 32.8 |
| | (40.3-40.5) | (35.7-37.8) | (32.6-37.0) | (34.2-39.9) | (34.8-43.9) | (33.8-35.3) | (25.6-40.9) |
| | | p < 0.0001 | p < 0.0001 | p < 0.0001 | p = 0.25 NS | p < 0.0001 | p < 0.0001 |
| Diabetes | | | | | | | |
| Yes | 0 | 3 | 0 | 1 | 0 | 0 | 0 |
| No | 346 | 83 | 7 | 9 | 2 | 65 | 2 |

There was no statistically significant difference between the cases and control groups in terms of time of serum sampling.

### Biomarkers

The serum levels of the biomarkers HbF, Hp, A1M, Hb-Total, Hp and Hpx are shown in Table 9.

Table 9 shows the mean concentrations with 95% confidence interval of the biochemical markers cell-free HbF, A1M, Hb-Total, Hp, Hpx and Uterine artery Doppler ultrasound Pulsatility Index (UtAD PI) Multiples of the Median (MoM). P-values were calculated with one-way ANOVA as compared to the control group. Analysis of the patient group's pregnancy induced hypertension and IUGR did not show any significant differences to the controls group.

| | | | Spontaneous |
|---|---|---|---|
| | Controls | Preeclampsia | preterm birth |
| | (n = 346) | (n = 86) | (n = 65) |
| Biomarker | (95% Cl) | (95% Cl) | (95% Cl) |
| HbF (μg/ml) | 5.6 | 10.8 | 3.5 |
| | (4.2-7.4) | (5.2-16.5) | (2.3-4.8) |
| | | p = 0.02 | p = 0.25 NS |
| A1M (μg/ml) | 15.5 | 17.3 | 14.1 |
| | (14.9-16.1) | (15.5-19.2) | (12.7-15.5) |
| | | p = 0.03 | p = 0.08 NS |
| Hb-Ttotal (μg/ml) | 297 | 258 | 201 |
| | (257-337) | (160-358) | (158-244) |
| | | p = 0.47 NS | p = 0.05 |
| Hp (μg/ml) | 971 | 1102 | 998 |
| | (915-1028) | (991-1131) | (863-1133) |
| | | p = 0.089 NS | p = 0.73 NS |
| Hpx (μg/ml) | 1143 | 1062 | 1061 |
| | (1111-1175) | (992-1132) | (992-1130) |
| | | p = 0.05 | p = 0.05 |
| UtAD PI MoM | 0.98 | 1.18 | 0.94 |
| | (0.92-0.99) | (1.04-1.31) | (0.87-1.02) |
| | | p < 0.0001 | p = 0.84 NS |

The mean concentration of HbF in the PE group (10.8 μg/ml, p=0.02) was significantly higher than in the control group (5.6 μg/ml). HbF is total HbF as compared to mainly non-complexed HbF described in Study I. The mean A1M concentration was also significantly increased (17.3 μg/ml vs. 15.5 μg/ml, p=0.03). The mean Hpx concentration in the PE group was significantly lower, 1062 μg/ml, compared to 1143 μg/ml in the control group (p=0.05). There was a tendency towards a slightly higher Hp concentration in the PE group (1102 μg/ml) as compared to the control group (971 μg/ml), however not significant (p=0.089). The PIH or IUGR showed comparable levels to the controls (data not shown). The SPTB group presented significantly lower levels of Hb-Total (201 μg/ml vs. 297 μg/ml, p=0.05) and Hpx (1061 μg/ml vs. 1143, p=0.05). The UtAD MoM values were significantly higher in the PE group than the controls (1.18 vs. 0.95 p<0.0001).

### Logistic Regression Analysis

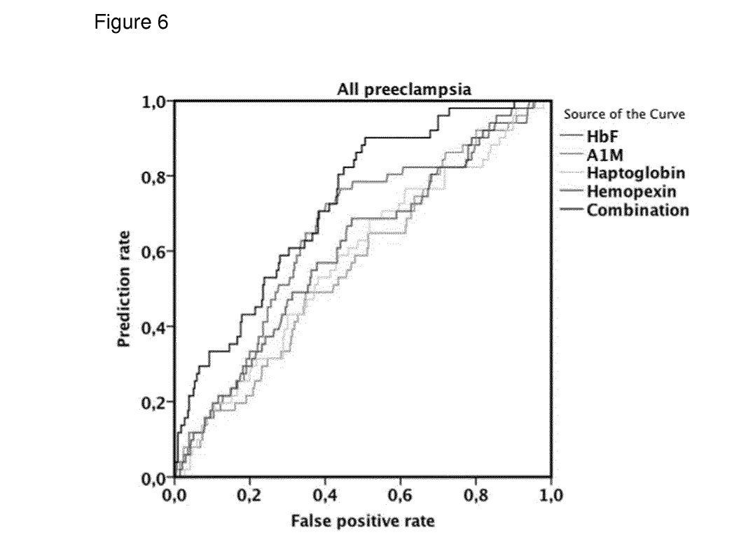

The abilities of the biomarkers to predict PE were tested in logistic regression models. Corresponding ROC-curves were generated to visualize the prediction values. All biomarkers were individually tested as well as evaluated in combination to find the optimal predictive value. The significant results are outlined in Table 10 and the ROC-curves are shown in FIG. 6.

TABLE 10
Prediction rates (PR) at different false positive rates (FPR) for each of the
different biomarkers, the UtAD Pulsatility (PI) MoM values and the maternal
characteristics. All prediction values are derived from ROC-curves based
on stepwise logistic regression models.

| | | | | | | Optimal |
|---|---|---|---|---|---|---|
| Model | AUC (95% CI) | 5% | 10% | 20% | 30% | (PR/FPR) |
| HbF* | 0.65 | 13% | 15% | 35% | 50% | 60%/35% |
| | (0.58-0.71) | | | | | |
| A1M* | 0.58 | 7% | 19% | 22% | 35% | 57%/46% |
| | (0.5-0.66) | | | | | |
| Hp £ | 0.58 | 9% | 17% | 30% | 49% | 53%/38% |
| | (0.5-0.66) | | | | | |
| Hpx* | 0.58 | 9% | 17% | 28% | 45% | 40%/28% |
| | (0.5-0.66) | | | | | |
| UtAD PI MoM | 0.60 | 18% | 25% | 39% | 49% | 48%/27% |
| | (0.52-0.68) | | | | | |
| HbF* + A1M* + | 0.73 | 22% | 33% | 43% | 59% | 66%/22% |
| Hp* + Hpx* | (0.66-0.8) | | | | | |
| Maternal characteristics*§ | 0.85 | 52% | 60% | 68% | 79% | 73%/23% |
| | (0.8-0.9) | | | | | |
| UtAD* + Maternal | 0.82 | 51% | 57% | 69% | 80% | 78%/27% |
| characteristics* | (0.75-0.89) | | | | | |
| UtAD* + Bimarkers* | 0.76 | 23 | 40 | 51 | 63 | 61%/24% |
| | (0.68-0.83) | 26% | 42% | 58% | 67% | |
| Biomarkers + | 0.83 | 60% | 62% | 68% | 81% | 81%/26% |
| Maternal characteristics*¶ | (0.75-0.91) | | | | | |
| Biomarkers + | 0.79 | 47% | 53% | 71% | 74% | 71%/19% |
| Maternal characteristics + | (0.71-0.87) | | | | | |
| UtAD | | | | | | |

Despite a significantly increased serum HbF concentration in patients who subsequently developed PE, it displayed limited predictive value when used alone (PR of 15% at FPR of 10%). A1M showed a similar prediction (PR of 19% at FPR of 10%). Hpx displayed the best individual prediction rates for PE (PR of 42% at FPR at 10%). The optimal prediction rate was obtained by combining A1M, HbF, and Hpx (PR of 62% at FPR of 10%).

All measures of maternal characteristics were tested alone and in combination using a logistic regression analysis to compare PE and controls.

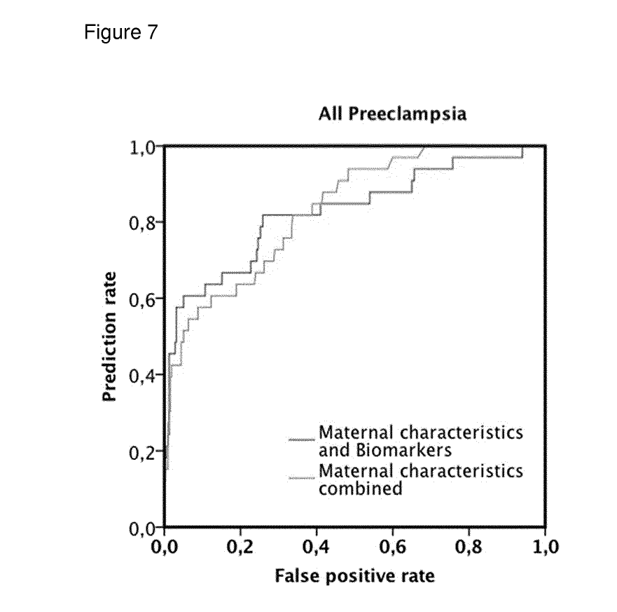

The combination of maternal characteristics (parity, diabetes, pre-pregnancy hypertension) and the biomarkers (HbF, A1M and Hpx) increased the PR to 62% at an FPR of 10% (Table 10, FIG. 7) and the combination UtAD and maternal characteristics combined showed a similar prediction rate (PR 57% at FPR 10%)

Early-vs. Late Onset Preeclampsia

We found elevated levels of HbF in both the early- and late onset PE groups (Table 11).

TABLE 11
The mean concentrations of biomarkers in the sub-groups early onset
PE (def.: delivery 34 + 0 weeks of gestation) and late onset PE (def.:
delivery >34 + 0 weeks of gestation). P-values were calculated with
one-way ANOVA as compared to the control group.

| | | Early onset | Late onset |
|---|---|---|---|
| | Controls | Preeclampsia | preeclampsia |
| | (n = 346) | (n = 16 (10)) | (n = 64) |
| Biomarker | (95% Cl) | (95% Cl) | (95% Cl) |
| HbF (μg/ml) | 5.6 | 13.7 | 10.1 |
| | (4.2-7.4) | (−6.8-34.2) | (4.8-15.4) |
| | | p = 0.05 | p = 0.04 |
| A1M (μg/ml) | 15.5 | 15.4 | 17.8 |
| | (14.9-16.1) | (12.5-18.4) | (15.6-20) |
| | | p = 0.98 NS | p = 0.01 |
| HbTotal (μg/ml) | 297 | 154 | 280 |
| | (257-337) | (67-241) | (162-399) |
| | | P = 0.23 NS | p = 0.78 NS |
| Hp (μg/ml) | 971 | 1108 | 1101 |
| | (915-1028) | (673-1542) | (943-1258) |
| | | P = 0.43 NS | p = 0.12 NS |
| Hpx (μg/ml) | 1143 | 947 | 1085 |
| | (1111-1175) | (757-1137) | (1009-1162) |
| | | p = 0.04 | p = 0.22 NS |
| UtAD PI MoM | 0.95 | 1.63 | 1.06 |
| | (0.92-0.99) | (1.2-2.06) | (0.94-1.19) |
| | | p < 0.00001 | p = 0.06 |

NS: not significant

The A1M levels were only significantly higher in the late onset group (p=0.01) (Table 11). The Hpx protein concentration was lower in both groups but only significant in the early onset PE group (p=0.04) (Table 11). UtAD PI MoM was significantly elevated especially in the early onset group (1.63 vs. 0.95, p<0.00001) but only marginally elevated in the late onset group and this difference was not statistically significant (1.06 vs. 0.95, p=0.06). There were no significant differences for Hb-Total or Hp in either of the study groups.

The logistic regression models for early- and late onset PE for the examined biomarkers showed a prediction rate for HbF of 23% at an FPR of 10%—but only in the late onset PE group. A1M was only statistically significant in the late onset group (p=0.01) and Hpx was only statistically significant for the early onset group and showed a PR of 32% at a FPR of 10%.

UtAD performed best in the early onset group with a PR of 57% at FPR 10% but was even statistically significant in the late onset group.

None of the biomarkers were statistically significant in combination with each other, with maternal characteristics or UtAD in either of the early- or late onset groups.

### Discussion

The aim of this study was to validate previous findings indicating that serum levels of cell-free HbF and A1M are elevated already in the first trimester of pregnancy and that they are useful as predictive first trimester biomarkers for the subsequent development of PE. The cohort size in this study is larger and reflects the normal incidence of PE better. In addition, the study also evaluates impact of the biologically related heme- and Hb-scavenging proteins Hp and Hpx.

The main finding in this paper confirms that both HbF and A1M are significantly elevated in serum from pregnant women who subsequently develop PE (Table 9). The increased serum concentrations of HbF are probably caused by a defect placental hematopoiesis reflecting placental oxidative stress. The data indicate that HbF and A1M have a potential as predictive first and early second trimester biomarkers for PE. Furthermore, the heme scavenger Hpx also show good predictive values and is therefore also suggested as an additional potential biomarker for PE. The UtAD indices primarily showed higher PI MoM values in the early onset group. This is in full concordance with previously published results from several research groups. The higher PI in the early onset group reflects increased resistance in the uterine arteries as a result of shallow invasion of the maternal decidual spiral arteries—a hallmark of early onset PE, but less common in late onset PE.

Interestingly, data showed that cell-free Hb-Total and Hpx were significantly lowered in patients who delivered prematurely. Low enzymatic activity of Hpx is known to attenuate endothelial inflammation. Lower levels of Hpx could therefore contribute to the increased maternal inflammation seen in both PE and preterm birth. Future studies are needed to more carefully decipher the role of Hpx in prematurity.

Specific first trimester screening for adverse pregnancy outcome is very important as it gives clinicians a tool to target and individualize surveillance of the patients rather than general screening programs later in pregnancy. By identifying high-risk pregnancies, preventive strategies and prophylactic treatment can be initiated. Up to date, the only prophylactic treatment is low dose acetyl salicylic acid (ASA). If the treatment is initiated before 16 weeks of gestation there is a markedly risk reduction (RR=0.47) especially for early onset and severe PE. The number needed to treat (NNT) may be as low as 7 for preventing severe PE in identified high-risk pregnancies. The use of ASA is cheap and has few side effects when given in the low doses recommended (75 mg). The prophylactic treatment should be initiated at the end of first trimester to have the optimal effect. In view of this, it is preferable if PE can be predicted at the end of first trimester or in the beginning of second trimester, possibly combined with other established screening programs for Down's syndrome.

### CONCLUSIONS

HbF, A1M and Hpx measured in maternal serum at the end of first and early second trimester of pregnancy are potential predictive biomarkers for subsequent development of PE. The three proteins are physiologically relevant, since increased amounts of cell-free HbF have been described to be involved in pathogenesis, and potentially consumes the physiological heme-scavenging proteins. Furthermore, the prediction power of the three biomarkers is increased by combination with uterine artery Doppler ultrasound and/or maternal characteristics.

### REFERENCES

1. Duley L. The global impact of pre-eclampsia and eclampsia. Semin Perinatol. 2009; 33(3):130-137.
2. Walker J J. Pre-eclampsia. Lancet. 2000; 356(9237):1260-1265.
3. Redman C W, Sargent I L. Latest advances in understanding preeclampsia. Science. 2005; 308(5728):1592-1594.
4. Roberts J M, Hubei C A. The two stage model of preeclampsia: variations on the theme. Placenta. 2009; 30 Suppl A:532-37.
5. Anderson U D, Olsson M G, Kristensen K H, Akerstrom B, Hansson S R. Review: Biochemical markers to predict preeclampsia. Placenta. 2012; 33 Suppl:542-47.
6. Rana S, Karumanchi S A, Lindheimer M D. Angiogenic factors in diagnosis, management, and research in preeclampsia. Hypertension. 2014; 63(2):198-202.
7. Hawkins T L, Roberts J M, Mangos G J, Davis G K, Roberts L M, Brown M A. Plasma uric acid remains a marker of poor outcome in hypertensive pregnancy: a retrospective cohort study. BJOG. 2012; 119(4):484-492.
8. Muller-Deile J, Schiffer M. Preeclampsia from a renal point of view: Insides into disease models, biomarkers and therapy. World J Nephrol. 2014; 3(4):169-181.
9. Hansson S R, Gram M, Åkerström B. Fetal hemoglobin in preeclampsia: A new etiological factor, a tool for predicting/diagnosis, and a potential target for therapy. Curr Opin Obstet Gynecol. 2013; Epub ahead of print.
10. Centlow M, Carninci P, Nemeth K, Mezey E, Brownstein M, Hansson S R. Placental expression profiling in preeclampsia: local overproduction of hemoglobin may drive pathological changes. Fertil Steril. 2008; 90(5):1834-1843.
11. Olsson M G, Centlow M, Rutardóttir S, et al. Increased levels of cell-free hemoglobin, oxidation markers, and the antioxidative heme scavenger a₁-microglobulin in preeclampsia. Free Radic Biol Med. 2010; 48(2):284-291.
12. Anderson U D, Olsson M G, Rutardóttir S, et al. Fetal hemoglobin and a₁-microglobulin as first- and early second-trimester predictive biomarkers for preeclampsia. Am J Obstet Gynecol. 2011; 204(6):520 e521-525.
13. May K, Rosenlöf L, Olsson M G, et al. Perfusion of human placenta with hemoglobin introduces preeclampsia-like injuries that are prevented by a₁-microglobulin. Placenta. 2011; 32(4):323-332.
14. Bunn H F. Hemoglobin. Vol. 1^{st} Installment. Weinhem: VCH; 1992.
15. Faivre B, Menu P, Labrude P, Vigneron C. Hemoglobin autooxidation/oxidation mechanisms and methemoglobin prevention or reduction processes in the bloodstream. Literature review and outline of autooxidation reaction. Artif Cells Blood Substit Immobil Biotechnol. 1998; 26(1):17-26.
16. Winterbourn C C. Oxidative reactions of hemoglobin. Methods Enzymol. 1990; 186:265-272.
17. Cimen M Y. Free radical metabolism in human erythrocytes. Clin Chim Acta. 2008; 390(1-2):1-11.
18. Jozwik M, Szczypka M, Gajewska J, Laskowska-Klita T. Antioxidant defence of red blood cells and plasma in stored human blood. Clin Chim Acta. 1997; 267(2):129-142.
19. Schaer D J, Buehler P W, Alayash A I, Belcher J D, Vercellotti G M. Hemolysis and free hemoglobin revisited: exploring hemoglobin and hemin scavengers as a novel class of therapeutic proteins. Blood. 2013; 121(8):1276-1284.
20. Alayash A I. Haptoglobin: Old protein with new functions. Clin Chim Acta. 2011; 412(7-8):493-498.
21. Kristiansen M, Graversen J H, Jacobsen C, et al. Identification of the haemoglobin scavenger receptor. Nature. 2001; 409(6817):198-201.
22. Polticelli F, Bocedi A, Minervini G, Ascenzi P. Human haptoglobin structure and function—a molecular modelling study. FEBS J. 2008; 275(22):5648-5656.
23. Ascenzi P, Bocedi A, Visca P, et al. Hemoglobin and heme scavenging. IUBMB Life. 2005; 57(11):749-759.
24. Delanghe J R, Langlois M R. Hemopexin: a review of biological aspects and the role in laboratory medicine. Clin Chim Acta. 2001; 312(1-2):13-23.
25. Hvidberg V, Maniecki M B, Jacobsen C, Hojrup P, Moller H J, Moestrup S K. Identification of the receptor scavenging hemopexin-heme complexes. Blood. 2005; 106(7):2572-2579.
26. Tenhunen R, Marver H S, Schmid R. The enzymatic conversion of heme to bilirubin by microsomal heme oxygenase. Proc Natl Acad Sci USA. 1968; 61(2):748-755.
27. Wagener F A, Eggert A, Boerman O C, et al. Heme is a potent inducer of inflammation in mice and is counteracted by heme oxygenase. Blood. 2001; 98(6):1802-1811.
28. Allhorn M, Berggård T, Nordberg J, Olsson M L, Åkerström B. Processing of the lipocalin a₁-microglobulin by hemoglobin induces heme-binding and heme-degradation properties. Blood. 2002; 99(6):1894-1901.
29. Larsson J, Allhorn M, Åkerström B. The lipocalin a₁-microglobulin binds heme in different species. Arch Biochem Biophys. 2004; 432(2):196-204.
30. Allhorn M, Klapyta A, Δkerström B. Redox properties of the lipocalin a₁-microglobulin: reduction of cytochrome c, hemoglobin, and free iron. Free Radic Biol Med. 2005; 38(5):557-567.
31. Δkerström B, Maghzal G J, Winterbourn C C, Kettle A J. The lipocalin a₁-microglobulin has radical scavenging activity. J Biol Chem. 2007; 282(43):31493-31503.
32. Olsson M G, Olofsson T, Tapper H, Δkerström B. The lipocalin a₁-microglobulin protects erythroid K562 cells against oxidative damage induced by heme and reactive oxygen species. Free Radic Res. 2008; 42(8):725-736.
33. Grubb A O, Lopez C, Tejler L, Mendez E. Isolation of human complex-forming glycoprotein, heterogeneous in charge (protein HC), and its IgA complex from plasma. Physiochemical and immunochemical properties, normal plasma concentration. J Biol Chem. 1983; 258(23):14698-14707.
34. Berggård T, Thelin N, Falkenberg C, Enghild J J, Δkerström B. Prothrombin, albumin and immunoglobulin A form covalent complexes with a₁-microglobulin in human plasma. Eur J Biochem. 1997; 245(3):676-683.
35. Coligan J E. New York: Wiley-Interscience; 1991.
36. Schaer D J, Vinchi F, Ingoglia G, Tolosano E, Buehler P W. Haptoglobin, hemopexin, and related defense pathways-basic science, clinical perspectives, and drug development. Front Physiol. 2014; 5:415.
37. Baek J H, D′Agnillo F, Vallelian F, et al. Hemoglobin-driven pathophysiology is an in vivo consequence of the red blood cell storage lesion that can be attenuated in guinea pigs by haptoglobin therapy. J Clin Invest. 2012; 122(4):1444-1458.
38. Reiter C D, Wang X, Tanus-Santos J E, et al. Cell-free hemoglobin limits nitric oxide bioavailability in sickle-cell disease. Nat Med. 2002; 8(12):1383-1389.
39. Minneci P C, Deans K J, Zhi H, et al. Hemolysis-associated endothelial dysfunction mediated by accelerated NO inactivation by decompartmentalized oxyhemoglobin. J Clin Invest. 2005; 115(12):3409-3417.
40. Wester-Rosenlöf L, Casslen V, Axelsson J, et al. A1M/alpha-1-microglobulin protects from heme-induced placental and renal damage in a pregnant sheep model of preeclampsia. PLoS One. 2014; 9(1):e86353.
41. Sverrisson K, Axelsson J, Rippe A, et al. Extracellular fetal hemoglobin induces increases in glomerular permeability: inhibition with alpha-1-microglobulin and tempol. Am J Physiol Renal Physiol. 2014; 306(4):F442-448.
42. Di Santo S, Sager R, Andres A C, Guller S, Schneider H. Dual in vitro perfusion of an isolated cotyledon as a model to study the implication of changes in the third trimester placenta on preeclampsia. Placenta. 2007; 28 Suppl A:S23-32.
43. Thatcher C D, Keith J C, Jr. Pregnancy-induced hypertension: development of a model in the pregnant sheep. Am J Obstet Gynecol. 1986; 155(1):201-207.
44. Talosi G, Nemeth I, Nagy E, Pinter S. The pathogenetic role of heme in pregnancy-induced hypertension-like disease in ewes. Biochem Mol Med. 1997; 62(1):58-64.
45. Laurell C B, Nyman M. Studies on the serum haptoglobin level in hemoglobinemia and its influence on renal excretion of hemoglobin. Blood. 1957; 12(6):493-506.
46. Olsson M G, Allhorn M, Bulow L, et al. Pathological conditions involving extracellular hemoglobin: molecular mechanisms, clinical significance, and novel therapeutic opportunities for alpha-1-microglobulin. Antioxid Redox Signal. 2012; 17(5):813-846.
47. Allhorn M, Lundqvist K, Schmidtchen A, Δkerström B. Heme-scavenging role of a₁-microglobulin in chronic ulcers. J Invest Dermatol. 2003; 121(3):640-646.
48. Olsson M G, Rosenlof L W, Kotarsky H, et al. The radical-binding lipocalin A1M binds to a Complex I subunit and protects mitochondrial structure and function. Antioxid Redox Signal. 2013; 18(16):2017-2028.
49. Olsson M G, Allhorn M, Larsson J, et al. Up-regulation of A1M/a₁-microglobulin in skin by heme and reactive oxygen species gives protection from oxidative damage. PLoS One. 2011; 6(11):e27505.
50. Tranquilli A L, Dekker G, Magee L, et al. The classification, diagnosis and managements of the hypertensive disordes of pregnancy: A revised statement from ISSHP. Pregnancy Hypertens. 2014; 4:97-104.
51. Brown M A. Pre-eclampsia: proteinuria in pre-eclampsia-does it matter any more? Nat Rev Nephrol. 2012; 8(10):563-565.
52. Gynecologists A-ACoOa. Hypertension in pregnancy; 2013.
53. Krikken J A, Lely A T, Bakker S J, et al. Hemopexin activity is associated with angiotensin II responsiveness in humans. J Hypertens. 2013; 31(3):537-541; discussion 542.
54. Bakker W W, Spaans F, el Bakkali L, et al. Plasma hemopexin as a potential regulator of vascular responsiveness to angiotensin II. Reprod Sci. 2013; 20(3):234-237.
55. Kajita A, Taniguchi K, Shukuya R. Isolation and properties of the gamma chain from human fetal hemoglobin. Biochim Biophys Acta. 1969; 175(1):41-48.
56. Noble R W. The effect of p-hydroxymercuribenzoate on the reactions of the isolated gamma chains of human hemoglobin with ligands. J Biol Chem. 1971; 246(9):2972-2976.
57. Δkerström B, Bratt T, Enghild J J. Formation of the alpha-1-microglobulin chromophore in mammalian and insect cells: a novel post-translational mechanism? FEBS Lett. 1995; 362(1):50-54.
58. Berggard I, Beam A G. Isolation and properties of a low molecular weight beta-2-globulin occurring in human biological fluids. J Biol Chem. 1968; 243(15):4095-4103.
59. Nilson B, Åkerström B, Logdberg L. Cross-reacting monoclonal anti-a₁-microglobulin antibodies produced by multi-species immunization and using protein G for the screening assay. J Immunol Methods. 1987; 99(1):39-45.
60. Bjorck L, Cigen R, Berggard B, Low B, Berggard I. Relationships between beta2-microglobulin and alloantigens coded for by the major histocompatibility complexes of the rabbit and the guinea pig. Scand J Immunol. 1977; 6(10):1063-1069.
61. Moller H J, Peterslund N A, Graversen J H, Moestrup SK. Identification of the hemoglobin scavenger receptor/CD163 as a natural soluble protein in plasma. Blood. 2002; 99(1):378-380.
62. Moestrup S K, Moller H J. CD163: a regulated hemoglobin scavenger receptor with a role in the anti-inflammatory response. Ann Med. 2004; 36(5):347-354.
63. Van Gorp H, Delputte P L, Nauwynck H J. Scavenger receptor CD163, a Jackof-all-trades and potential target for cell-directed therapy. Mol Immunol. 2010; 47(7-8):1650-1660.
64. Velauthar L, Plana M N, Kalidindi M, et al. First-trimester uterine artery Doppler and adverse pregnancy outcome: a meta-analysis involving 55,974 women. Ultrasound Obstet Gynecol. 2014; 43(5):500-507.
65. Malek M H, Berger D E, Coburn J W. On the inappropriateness of stepwise regression analysis for model building and testing. Eur J Appl Physiol. 2007; 101(2):263-264; author reply 265-266.

1-32. (canceled)

33. A method for diagnosing, predicting, or evaluating the risk of developing preeclampsia in a pregnant female mammal, comprising measuring levels of hemopexin (Hpx) and alpha-1-microglobulin (A1M) in a biological sample from the pregnant female mammal.

34. The method of claim 33, wherein the pregnant female mammal is a human, further comprising comparing the measured levels of Hpx and A1M to reference values for Hpx and A1M, wherein the reference values for Hpx and A1M are derived from levels of Hpx and A1M measured in biologicals samples from reference pregnant women who do not develop preeclampsia.

35. The method of claim 34, further comprising determining that the subject has preeclampsia or it at increased risk of developing preeclampsia when the measured level of Hpx is at least 1.1 times less than the reference value for Hpx, the measured level of A1M is at least 1.1 times more than the reference value for A1M.

36. The method of claim 33, wherein the biological sample is selected from blood, plasma, serum, cerebrospinal fluid, urine, placental biopsies, uterine fluid and amniotic fluid.

37. The method of claim 33, wherein the method is for diagnosing, predicting, or evaluating the risk of developing early onset preeclampsia.

38. The method of claim 33, wherein the pregnant female mammal is a human and the biological sample is taken from the subject at a gestational age of 6-20 weeks.

39. The method of claim 33, wherein the pregnant female mammal is a human and the biological sample is taken from the subject at a gestational age of 12-14 weeks.

40. The method of claim 33, wherein the pregnant female mammal is a human and the biological sample is a plasma sample and is taken from the subject at a gestational age of 6-20 weeks, further comprising determining that the subject has preeclampsia or it at increased risk of developing preeclampsia when the measured level Hpx in the plasma sample is 1.0 mg/mL or less, and the measured level of A1M in the serum sample is 15.5 μg/mL or more.

41. The method of claim 33, wherein the pregnant female mammal is a human and the method is for diagnosing, predicting, or evaluating the risk of developing late onset preeclampsia.

42. The method of claim 33, wherein the pregnant female mammal is a human and the biological sample is taken from the subject at a gestational age of 34-40 weeks.

43. The method of claim 33, wherein the pregnant female mammal is a human and the biological sample is a plasma sample and is taken from the subject at a gestational age of 34-40 weeks, further comprising determining that the subject has preeclampsia or it at increased risk of developing preeclampsia when the measured level Hpx in the plasma sample is 0.85 mg/mL or less, and the measured level of A1M in the serum sample is 30 μg/mL or more.

44. A method for the diagnosis or aiding in the diagnosis of preeclampsia, comprising:
(a) obtaining a biological sample from a pregnant female mammal;
(b) measuring the levels of hemopexin (Hpx) and alpha-1-microglobulin (A1M) in the sample; and
(c) comparing the measured levels of Hpx and A1M with a reference value to determine if the a pregnant female mammal has or has not preeclampsia, or is or is not at increased risk of developing preeclampsia.

45. The method of claim 44, wherein the biological sample is taken at a gestational age of 6-20 weeks.

46. The method of claim 44, wherein the biological sample is taken at a gestational age of 34-40 weeks.

47. A method for monitoring the progression or regression of preeclampsia a pregnant female mammal, comprising:
(a) measuring the levels of hemopexin (Hpx) and alpha-1-microglobulin (A1M) in a first biological sample from a pregnant female mammal;
(b) measuring the levels of hemopexin (Hpx) and alpha-1-microglobulin (A1M) in a second biological sample obtain from the pregnant female mammal at a later time than the first sample;
(c) comparing the values measured in step (a) and (b), and
(i) determining that preeclampsia is progressing in the pregnant female mammal when the measured Hpx level in the second biological sample is lower than the measured Hpx level in the first biological sample and the measured A1M level in the second biological sample is higher than the measured A1M level in the first biological sample; or
(ii) determining that preeclampsia is regressing in the pregnant female mammal when the measured Hpx level in the second biological sample is higher than the measured Hpx level in the first biological sample and the measured A1M level in the second biological sample is lower than the measured A1M level in the first biological sample.

48. The method of claim 47, wherein the biological sample is selected from blood, plasma, serum, cerebrospinal fluid, urine, placental biopsies, uterine fluid and amniotic fluid.

49. The method of claim 47, wherein the first biological sample is taken at a gestational age of at least 6 weeks.

50. The method of claim 47, wherein the first biological sample is taken at a gestational age of 6-20 weeks.

51. The method of claim 47, wherein the first biological sample is taken at a gestational age of 12-14 weeks.

52. The method of claim 47, wherein the first biological sample is taken at a gestational age of 34-40 weeks.
